(19) United States
(12) Patent Application Publication
(10) Pub. No.: US 2005/0049198 A1
(43) Pub. Date: Mar. 3, 2005

(54) Method of modulating the activity of calcium channels in cardiac cells and reagents therefor

(76) Inventors: Angela F. Dulhunty, Queanbeyan (AU); Marco G. Casarotto, Kaleen (AU)

Correspondence Address: PILLSBURY WINTHROP LLP, ATTENTION: DOCKETING DEPARTMENT, 11682 EL CAMINO REAL, SUITE 200, SAN DIEGO, CA, 92130, US

(21) Appl. No.: 10/478,112

(22) PCT Filed: May 17, 2002
(86) PCT No.: PCT/AU02/00608

(30) Foreign Application Priority Data
May 17, 2001 (AU) PR 5066

(51) Int. Cl. A61K 38/10

(52) U.S. Cl. 514/13

(57) ABSTRACT

The present invention relates generally to novel peptides that are capable of modulating the activity of calcium channels in cardiac cells. More specifically, the present invention provides a method of modulating the activity of a cardiac calcium channel comprising contacting a cardiac ryanodine receptor (RyR2) with an amount of a fragment of a dihydropyridine receptor (DHPR) polypeptide sufficient to modulate the activity of said RyR2, and determining the activity of said calcium channel. The inventive method is useful for the treatment of a range of disorders and diseases associated with cardiac dysfunction, particularly those diseases and disorders involving reduced cardiac output and/or aberrant excitation-contraction coupling, calcium overload, or calcium leakage, in cardiac cells.

### FIELD OF THE INVENTION

The present invention relates generally to novel peptides that are capable of modulating the activity of calcium channels in cardiac cells. More specifically, the present invention provides a method of modulating the activity of a cardiac calcium channel comprising contacting a cardiac ryanodine receptor (RyR2) with an amount of a fragment of a dihydropyridine receptor (DHPR) polypeptide sufficient to modulate the activity of said RyR2, and determining the activity of said calcium channel. The inventive method is useful for the treatment of a range of disorders and diseases associated with cardiac dysfunction, particularly those diseases and disorders involving reduced cardiac output and/or aberrant excitation-contraction coupling, calcium overload, or calcium leakage, in cardiac cells.

### BACKGROUND TO THE INVENTION

Bibliographic details of the publications referred to in this specification are collected at the end of the description. Reference herein to prior art, including any one or more prior art documents, is not to be taken as an acknowledgment, or suggestion, that said prior art is common general knowledge in Australia or forms a part of the common general knowledge in Australia.

Excitation-contraction coupling is essential to the functioning of striated muscles, such as cardiac and skeletal muscles, linking electrical excitation to mechanical activity. Key components of excitation-contraction coupling are the dihydropyridine receptor (DHPR), a voltage-dependent Ca²⁺ channel of the transverse tubule (TT), and the Ca²⁺ release channel or ryanodine receptor (RyR) of the sarcoplasmic reticulum (SR) membrane that opens to release calcium from the SR into the cytoplasm.

Different isoforms of both RyRs and DHPRs exist in cardiac and skeletal muscle cells. In particular, the RyR1 and DHPR-isoform 3 are predominant in skeletal muscle and the RyR2 and DBPR-isoform 1 are predominant in cardiac cells. There is only about 70% identity between RyR1 and RyR2 amino acid sequences.

In skeletal muscle, it is known that the ryanodine receptor (RyR1) is activated by a protein-protein interaction with a 138 amino acid cytoplasmic loop between repeats II and III of the DHPR α-1 subunit (Tanabe et al., 1990, Nature 346:567-568). A region of the skeletal DHPR cytoplasmic loop that is sufficient to activate skeletal muscle RyR1-mediated calcium release has been determined to reside within 20 amino acids from Thr⁶⁷¹ to Leu⁶⁹⁰ (El Hayek et al., 1995, J. Biol. Chem. 270:22116-22118; Dulhunty et al., 1999, Biophys. J. 77:189-203; and Gurrola et al., 1999, J. Biol. Chem. 274:7879-7886). There are four DHPR molecules located in a tetrad configuration of DHPRs opposite every second RyR1 polypeptide in skeletal muscle, in a strict geometrical alignment that is considered to be important for normal excitation-contraction coupling. During excitation of the DHPR, such as by electrical stimulation, this protein-protein interaction presumably induces a conformational shift in the RyR1 polypeptide that results in channel opening, thereby causing calcium efflux from the SR. Accordingly, excitation-contraction coupling in skeletal muscle is essentially a calcium-independent process.

In contrast, excitation-contraction coupling in cardiac muscle involves a calcium-induced calcium release (CICR) mechanism (Niggli, 1999, Annu Rev Physiol 61:311-335). The opening of cardiac DHPR calcium channels following their excitation results in a small amount of extracellular calcium influx into cardiac myocytes via the voltage-dependent L-type calcium channels (i.e. cardiac DHPRs) that are activated during each action potential. This initial signal acts as a trigger for subsequent CICR from the intracellular calcium stores in the SR. The secondary calcium release from the SR occurs via the cardiac RyR2 calcium release channels. In most mammals, CICR amplifies the initial signal trigger several-fold, consistent with the stoichiometry of cardiac RyR2 molecules to cardiac DHPR of about 16:1.

Notwithstanding that cardiac CICR requires an initial elevation of cytosolic Ca²⁺ to trigger calcium release from the SR, it does not appear to become self-sustaining. This is because CICR possibly propagates only between cardiac myocytes that are overloaded with calcium, however is normally localized to individual cells. Additionally, the force of cardiac muscle contraction increases proportionately with [Ca²⁺]ᵢ between about 3×10⁻⁷M [Ca²⁺]ᵢ and about 10⁻⁶M [Ca²⁺]_{I}, suggesting that CICR is not an all or nothing response.

In contrast to excitation-contraction coupling in skeletal myocytes, it is not known whether or not there is any direct protein-protein interaction between cardiac DHPR and cardiac RyR2 in vivo, however the cytoplasmic loop of the skeletal DHPR α-1 subunit does bind to cardiac RyR2 in two hybrid assays (Osterland et al., 1999, Biophys. J. 76:A467-(abstract)) and the 20-mer peptide (i.e. Thr⁶⁷¹ to Leu⁶⁹⁰) of the skeletal DHPR α-1 subunit cytoplasmic loop binds to RyR2 in surface plasmon resonance studies (O'Reilly and Ronjat, 1999, Biophys. J. 76:A466-(abstract)).

Compelling data indicate that the relationship between skeletal and cardiac DHPRs and RyRs channels is different. For example, cardiac RyR2 expressed in dyspedic myocytes that lack skeletal RyR1 but contain skeletal DHPR α-1 subunit, cannot support skeletal type excitation-contraction coupling. Additionally, isolated RyR2 channels are not activated by the entire 138-amino acid cytoplasmic loop between repeats II and III of cardiac or skeletal DHPR α-1 subunits (Lu et al., 1994, J. Biol. Chem. 269:6511-6516). Additionally, the 20-mer peptide (i.e. Thr⁶⁷¹ to Leu⁶⁹⁰) of the skeletal DHPR α-1 subunit cytoplasmic loop has been shown not to induce Ca²⁺ release from cardiac SR, or to enhance [³H]ryanodine binding to cardiac RyR2 channels (El Hayek, et al., 1995, supra), despite the fact that it is a high affinity activator of skeletal muscle RyR1 channels. Moreover, these available data suggest that the 20-mer peptide (i.e. Thr⁶⁷¹ to Leu⁶⁹⁰) of the skeletal DHPR α-1 subunit cytoplasmic loop cannot activate cardiac RyR2 channels, such as, for example, by prolonging their open time or frequency of opening.

Stern (1992, FASEB J. 6:3092-3100) proposed that Ca²⁺ synapses (i.e. localized domains of very high Ca²⁺ near the site of Ca²⁺ entry and release) functionally link the activities of DHPRs and RyRs in cardiac tissue. Each Ca²⁺ synapse allows local control of the RyR2 by virtue of the high local Ca²⁺ concentration, so as to produce the observed high signal amplification without spreading of the Ca²⁺ release signal across the entire cell or between cells. As a consequence, the number of release units recruited during each signal could be quantitatively graded with the trigger calcium release. This proposal is consistent with the observation of calcium sparks of short duration (about 50 ms) and limited spatial spread (about 1.5 μm) during stimulation of cardiac myocytes (Cheng et al., 1993, Science 262:740-744).

Myocardial contractile failure is a common cause of morbidity and mortality in patients with ischemic heart disease, congestive heart failure, and systemic inflammatory states such as sepsis. Accumulating evidence indicates that contractile failure is associated with dysregulation of myoplasmic calcium flux. Reduced Ca²⁺ sensitivity of the myofilaments or a deterioration of calcium signalling, such as, for example, by deterioration or disruption of the calcium synapse, deterioration of the RyR2, or deterioration of the DHPR, may lead to a decline in the force of cardiac contractions. Several Ca²⁺ signal pathways are adversely affected during cardiac failure or cardiac hypertrophy (with calcium overload observed in end-stage heart failure). An elevated resting Ca²⁺ concentration, reduced Ca²⁺ transient amplitude, slowed relaxation, and altered Ca²⁺ pump in the SR have been observed in failing or hypertrophic cardiac tissue.

More particularly, hypertrophied as well as failing hearts show decreased excitation-contraction coupling efficiencies compared to normal hearts (Gomez et al., 1997, Science 276:755-756). However, individual DHPRs and RyRs in failing or hypertrophic hearts appear normal, suggesting that the link between these two calcium signal proteins may be defective. This view is supported by the restoration of normal excitation-contraction coupling in hypertrophic cells or following congestive heart failure by the application of β-adrenergic agonists to prolong the open time of cardiac DHPRs.

Additionally, hyperphosphorylation of RyR2 in failing human hearts results in defective channel function.

In addition, cardiac output can be boosted in acute situations, such as after heart attack, by “inotropic agents” that enhance excitation-contraction coupling. Discrete areas of heart muscle are damaged during ischaemic episodes, causing reduced cardiac output. Blood supply to essential organs such as the brain can be maintained by asking the remaining healthy heart muscle to contract more strongly. This is currently done by drugs which mimic beta adrenergic stimulation and increase cAMP levels to stimulate DHPR activity and excitation-contraction coupling. In the long term increased cAMP levels can become toxic and lead to calcium overload.

### SUMMARY OF THE INVENTION

This specification contains nucleotide and amino acid sequence information prepared using the program PatentIn Version 3.1, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by descriptor “SEQ ID NO:” followed by the numeric identifier. For example, SEQ ID NO: 1 refers to the information provided in the numeric indicator field designated <400>1, etc.

Reference herein to the consensus sequence set forth in SEQ ID NO: 1 shall be taken to include a reference to any one or more of the amino acid sequences set forth in SEQ ID Nos: 2-7 used to compile said consensus sequence.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used herein the term “derived from” shall be taken to indicate that a specified integer may be obtained from a particular source albeit not necessarily directly from that source.

In work leading up to the present invention, the inventors sought to identify novel means for modulating CICR in cardiac tissue, so as to provide for improved treatment regimes for cardiac failure and/or cardiac hypertrophy. Surprisingly, although no physical connection between cardiac DHPRs and cardiac RyRs has been established, the present invention provides small fragments of the skeletal or cardiac DHPR polypeptides, such as, for example, small basic charged peptides, that can activate cardiac RyR2 channels.

More particularly, the present inventors have shown that the 20-mer peptide (i.e. Thr⁶⁷¹ to Leu⁶⁹⁰) of the skeletal DHPR α-1 subunit cytoplasmic loop produces significant activation of RyR2 channels at −40 mV (negative potentials induce calcium release from the SR) and strong inhibition at +40 mV. Activation of cardiac RyR2 was observed at concentrations as low as 1 nM peptide, significantly less than the peptide concentration required to activate skeletal muscle RyR1. Additionally, the inhibition of cardiac RyR2 channels was significantly greater than that observed for skeletal RyR1 channels at 3×10⁻⁷M cytoplasmic Ca²⁺.

Accordingly, one aspect of the present invention provides a method for modulating the activity of a cardiac ryanodine receptor (RyR2) calcium channel comprising contacting a cardiac RyR2 with an amount of a fragment of a dihydropyridine receptor (DHPR) polypeptide, such as, for example, a basic charged fragment, sufficient to modulate the activity of said RyR2.

More preferably, the present invention provides a method for modulating the activity of a cardiac ryanodine receptor (RyR2) calcium channel comprising contacting a cardiac RyR2 with an amount of a fragment of a dihydropyridine receptor (DHPR) polypeptide, such as, for example, a basic charged fragment, sufficient to modulate the activity of said RyR2, and determining the activity of said cardiac RyR2 calcium channel.

It will be apparent from the preceding discussion that one embodiment of the invention is directed to a method for enhancing the activity of a cardiac RyR2 calcium channel comprising contacting a cardiac RyR2 with an amount of a fragment of a dihydropyridine receptor (DHPR) polypeptide sufficient to enhance the activity of said RyR2, and determining the activity of said cardiac RyR2 calcium channel. As exemplified herein and without limiting the invention to any theory or mode of action or effective peptide concentration, the present inventors have shown that, for isolated cardiac RyR2 in a lipid bilayer, both the frequency of channel openings and the duration of each channel opening is enhanced by the application of up to about 1 nM peptide to about 10 μM peptide. At high peptide concentrations, the activity of channels that opened first declines slightly however the activity of other channels is more pronounced, presumably reflecting a microheterogeneity in RyR2 channel sensitivities to peptide.

It will also be apparent from the preceding discussion that another embodiment of the invention is directed to a method for inhibiting the activity of a cardiac RyR2 calcium channel comprising contacting a cardiac RyR2 with an amount of a fragment of a dihydropyridine receptor (DHPR) polypeptide sufficient to inhibit the activity of said RyR2, and determining that said cardiac RyR2 calcium channel lacks activity. As exemplified herein and without limiting the invention to any theory or mode of action or effective peptide concentration, the present inventors have shown that, for isolated cardiac RyR2 in a lipid bilayer, the frequency of channel openings particularly at +40 mV is reduced by a concentration in excess of about 10 μM peptide, possibly as a consequence of peptide binding within the pore of the RyR2 channel at a site that is distinct from the site at which it binds during channel activation.

A second aspect of the invention provides a method for identifying a peptide modulator of a cardiac RyR2 calcium channel comprising:
(i) incubating an amount of a fragment of a dihydropyridine receptor polypeptide or a homologue, analogue or derivative thereof that modulates cardiac RyR2 channel activity in the presence of a functional cardiac RyR2 calcium channel under conditions appropriate for calcium channel activity to be modulated and determining the activity of the channel;
(ii) incubating a candidate peptide in the presence of said functional cardiac RyR2 calcium channel under conditions appropriate for calcium channel activity to be modulated by said dihydropyridine receptor polypeptide or a homologue, analogue or derivative thereof and determining the activity of the channel;
(iii) comparing the activity at (i) and (ii); and
(iv) preferably selecting those peptides having comparable or enhanced modulation of channel activity at (ii) relative to (i).

In an alternative embodiment, this aspect of the invention provides a method for identifying a peptide modulator of a cardiac RyR2 calcium channel comprising:
(i) incubating an amount of a fragment of a dihydropyridine receptor polypeptide or a homologue, analogue or derivative thereof that modulates cardiac RyR2 channel activity in the presence of a functional cardiac RyR calcium channel under conditions appropriate for calcium channel activity to be modulated and determining the activity of the channel;
(ii) incubating a candidate peptide and an amount of said dihydropyridine receptor polypeptide or a homologue, analogue or derivative thereof that modulates cardiac RyR2 channel activity in the presence of a functional cardiac RyR2 calcium channel under conditions appropriate for calcium channel activity to be modulated by said dihydropyridine receptor polypeptide or a homologue, analogue or derivative thereof and determining the activity of the channel;
(iii) comparing the activity at (i) and (ii); and
(iv) preferably selecting those peptides having comparable or enhanced modulation of channel activity at (ii) relative to (i).

A third aspect of the present invention provides a method of determining whether a cardiac RyR2 channel is open or has a high channel open probability said method comprising contacting a cardiac RyR2 channel with an amount of a fragment of a dihydropyridine receptor (DHPR) polypeptide or homologue, analogue or derivative for a time and under conditions sufficient for binding to said channel to occur and determining the binding of said peptide to said channel, wherein binding of said peptide to said channel indicates a high channel open probability and wherein non-specific peptide binding indicates a low channel open probability.

A fourth aspect of the present invention provides a method of treatment of cardiac dysfunction in a human or animal subject comprising administering an effective amount of a fragment of a dihydropyridine receptor (DHPR) polypeptide or homologue, analogue or derivative for a time and under conditions sufficient for enhanced cardiac contraction to occur thereby rectifying said cardiac dysfunction.

A fifth aspect of the present invention provides a pharmaceutical composition comprising a fragment of a dihydropyridine receptor polypeptide, which peptide comprises at least about 5 contiguous amino acid residues of the peptide set forth in any one of SEQ ID NOs: 1-10 or a homologue, analogue or derivative thereof together with one or more pharmaceutically acceptable diluents.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue or as | Xaa | X |
| otherwise defined | | |

### BRIEF DESCRIPTION OF THE DRAWINGS

FIG. 1 is a schematic representation showing the aligned amino acid sequences of the cytoplasmic loops of several cardiac and skeletal DHPR polypeptides, as follows: human skeletal muscle DHPR-3 (SEQ ID NO: 2; Drouet et al., 1993); murine skeletal muscle DHPR-3 (SEQ ID NO: 3; Chaudhari, 1992); rabbit skeletal muscle DHPR-3 (SEQ ID NO: 4; Tanabe et al., 1987); rabbit cardiac DHPR-1 (SEQ ID NO: 5); rat cardiac DHPR-1 (SEQ ID NO: 6); and bullfrog skeletal muscle DHPR-3 (SEQ ID NO: 7; Zhou et al., 1998). A consensus sequence (SEQ ID NO: 1) is indicated in bold type, based upon a comparison of the available sequences. The amino acid sequences of the non-specific peptides designated NB (SEQ ID NO: 8) and A1S (SEQ ID NO: 9) are also indicated.

FIG. 2 is a graphical representation showing that a 20-mer peptide of the skeletal DHPR cytoplasmic loop (SEQ ID NO: 2) added to the cytoplasmic (cis) face of isolated cardiac RyR2 in a lipid bilayer increases the activity of cardiac RyR2 in the presence of cis 10⁻⁷M Ca²⁺ when measured at −40 mV. Single channel activity was determined at −40 mV, either in the absence of added peptide (Panel A), or in the presence of 65 nM peptide (Panel B), or 6.5 μM peptide (Panel C).

At the left hand side of each panel, downward channel opening is indicated at −40 mV, wherein zero current (closed) level is shown by the dotted line “C” and the maximum single channel conductance is shown by the continuous line “O”. Two channels are active in the bilayer at −40 mV after addition of peptide.

At the right hand side of each panel is a graphical representation of all points during a 30 s recording period. The numbers on the x-axis indicate the amplitude of channel opening and the abscissa indicates the proportion of total openings at each amplitude. Negative numbers indicate negative (inward) channel currents at 40 mV.

FIG. 3 is a graphical representation showing that a 20-mer peptide of the skeletal DHPR cytoplasmic loop (SEQ ID NO: 2) added to the cytoplasmic (cis) solution increases and then decreases the activity of cardiac RyR2 in the presence of cis 10⁻⁷M Ca²⁺ at +40 mV. Single channel activity was determined at +40 mV, either in the absence of added peptide (Panel A), or in the presence of 65 nM peptide (Panel B), or 32.5 μM peptide (Panel C).

At the left hand side of each panel, upward channel opening is indicated at +40 mV, wherein zero current (closed) level is shown by the dotted line “C” and the maximum single channel conductance is shown by the continuous line “O”. The channel is mostly in the open configuration at 65 nM concentration. In contrast, the channel is mostly in the closed configuration in the absence of peptide, or at 32.5 μM peptide.

At the right hand side of each panel is a graphical representation of all points during a 30 s recording period. The numbers on the x-axis indicate the amplitude of channel opening and the abscissa indicates the proportion of total openings at each amplitude. Numbers indicate channel opening at +40 mV.

FIG. 4 is a graphical representation showing the average normalized mean current (abscissa) as a function of peptide concentration (i.e. log₁₀[peptide (nM)]) in cytoplasmic solution (x-axis) at both −40 mV (Panel A) and at +40 mV (Panel B). Normalized mean current (I′p/I′c), is the ratio of the mean current in the presence of peptide (i.e. l′p) to the mean current in the absence of peptide under control conditions (i.e. l′c). The data indicate average mean current±SEM. Samples either contained the 20-mer peptide of the skeletal DHPR cytoplasmic loop set forth in SEQ ID NO: 2 (n=S, ●), or the non-specific peptide NB set forth in SEQ ID NO: 8 (n=2, ▴), or the non-specific peptide A1S set forth in SEQ ID NO: 9 (n=3, ▪). A normalized mean current of greater than 1.0 indicates activation of the cardiac RyR2 channel by peptide. Accordingly, data indicate significant activation of cardiac RyR2 channels by up to 10 μM peptide (SEQ ID NO: 2) at +40 mV, or higher concentrations at −40 mV.

FIG. 5 is a graphical representation of the subject peptides increasing Ca²⁺- and caffeine-activated Ca²⁺ release from cardiac SR vesicles. The initial rate of Ca²⁺ release is shown under control conditions or following addition of either 20 μM Ca²⁺ or with 2 mM caffeine (at zero peptide concentration) and after addition of peptide alone (small symbols), or after addition of 20 μM Ca²⁺ or 2 mM caffeine plus peptide (large symbols) to the extravesicular solution at the concentrations shown on the abscissa. The rate of Ca²⁺ release is given as μmoles of Ca²⁺, per mg of protein in the SR vesicles, per min. Data is shown for SEQ ID NO: 2 (filled circles), SEQ ID NO: 8 (filled squares), SEQ ID NO: 9 (open circles) and SEQ ID NO: 10 (open squares). Results are given as mean±sem with at least 5 observations for each concentration.

FIG. 6 is a graphical representation of the effects of SEQ ID NO: 9 on the mean current flowing through RyR channels incorporated into lipid bilayers, with a cis (cytoplasmic) Ca²⁺ concentration of 100 μM. Data was obtained from 60 s of recordings at a bilayer potential of −40 mV (upper graph) and +40 mV (lower graph). The data points show average relative mean current (mean current with peptide divided by mean current under control conditions) from six experiments and the vertical bars show ±1 sem. Channel activity is clearly depressed by the peptide in a concentration-dependent manner with maximum effects at 100 nM peptide. The depression at 100 nM peptide was significantly greater at +40 mV (asterisk) than at −40 mV.

FIG. 7 is a graphical representation of the effects of SEQ ID NO: 9 on the mean current flowing through RyR channels incorporated into lipid bilayers, with a cis (cytoplasmic) Ca²⁺ concentration of 100 nM. Data was obtained from 60 s of recordings at a bilayer potential of −40 mV (upper graph) and +40 mV (lower graph). The data points show average relative mean current (mean current with peptide divided by mean current under control conditions) from seven experiments and the vertical bars show ±1 sem. Channel activity is clearly enhanced by the peptide in a concentration-dependent manner with maximum effects at 10 nM peptide. Depression at >100 nM peptide was seen at +40 mV and is indicative of some pore block.

FIG. 8 is a graphical representation of the effects of SEQ ID NO: 9 on single channel parameters as a function of peptide concentration. Data is shown at −40 mV (left panel) and +40 mV (right panel). The top graphs show the open probability of the channels, the second graphs show mean open time, followed by mean closed time and finally the frequency of channel opening. The data points show average parameter values from seven experiments and the vertical bars show ±1 sem. The decrease in the closed time of the channel and consequent increase in frequency contribute most strongly to the increase in open probability.

### DETAILED DESCRIPTION OF THE PREFERRED EMBODIMENTS

One aspect of the present invention provides a method for modulating the activity of a cardiac ryanodine receptor (RyR2) calcium channel comprising contacting a cardiac RyR2 channel with an amount of a fragment of a dihydropyridine receptor (DHPR) polypeptide sufficient to modulate the activity of said RyR2.

More particularly, the present invention provides a method for modulating the activity of a cardiac ryanodine receptor (RyR2) calcium channel comprising contacting a cardiac RyR2 channel with an amount of a fragment of a dihydropyridine receptor (DHPR) polypeptide sufficient to modulate the activity of said RyR2, and determining the activity of said cardiac RyR2 calcium channel.

As used herein, the term “ryanodine receptor-2 channel” or “RyR2 channel” or “cardiac RyR channel” shall be taken to refer to a calcium channel that comprises RyR2 polypeptide subunits. Those skilled in the art are aware of the physical structure of a “RyR2 channel” or “cardiac RyR channel” referred to herein.

As used herein, the term “modulating” shall be taken to mean enhancing or inhibiting the activity of an RyR2 calcium channel. Accordingly, by “modifying the activity of an RyR2 calcium channel” or similar term is meant generally that CICR is modified (i.e. enhanced or reduced) such as, for example, by modifying the calcium synapse or calcium sensitivity of cardiac RyR2, the frequency of cardiac RyR2 channel openings during each action potential, or the open time of individual cardiac RyRs.

As stated supra the present invention clearly encompasses both a method for enhancing the activity of a cardiac RyR2 calcium channel and a method for inhibiting the activity of a cardiac RyR2 calcium channel.

The cardiac RyR2 channel may be in situ in a cardiac cell, or in cardiac muscle in vivo, however it may also be an isolated RyR2 channel, such as, for example, inserted in lipid bilayer, or alternatively, a recombinant or reconstituted RyR2 channel such as, for example, expressed in the membrane of a transfected cell (e.g. a CHO cell or dyspedic myocyte). Standard procedures, such as, for example, as described by Bhat et al. (1997, Biophys. J. 73:1329-1336), which is herein incorporated by reference, can be used to express the RyR2 channel in transfected cells. Preferably, the cardiac RyR2 channel is in situ in a cardiac cell or in vivo in cardiac tissue.

As used herein, the term “fragment” means that the peptide has an overall positive charge at physiological pH values by virtue of the presence of a relatively high proportion of basic amino acid residues, such as, for example, arginine or lysine, or the half-basic amino acid residue, histidine. Preferably, a fragment will have at least about 25% basic amino acid residues, and more preferably at least about 50% basic amino acid residues.

The peptide that is useful in performing the invention described herein is of at least about 5 amino acids in length from a cardiac or skeletal dihydropyridine receptor (DHPR) polypeptide fragment comprising the amino acid sequence TSAQKXXXEE (R/K)XR(R/K)K(M/L)(A/S)(R/K)XX (SEQ ID NO: 1), wherein X is any amino acid residue. The present invention clearly extends to the use of peptides having a similar sequence to SEQ ID NO: 1 from any source, such as, for example, a synthetic or naturally-occurring peptide, or a peptide derived from any DHPR sequence.

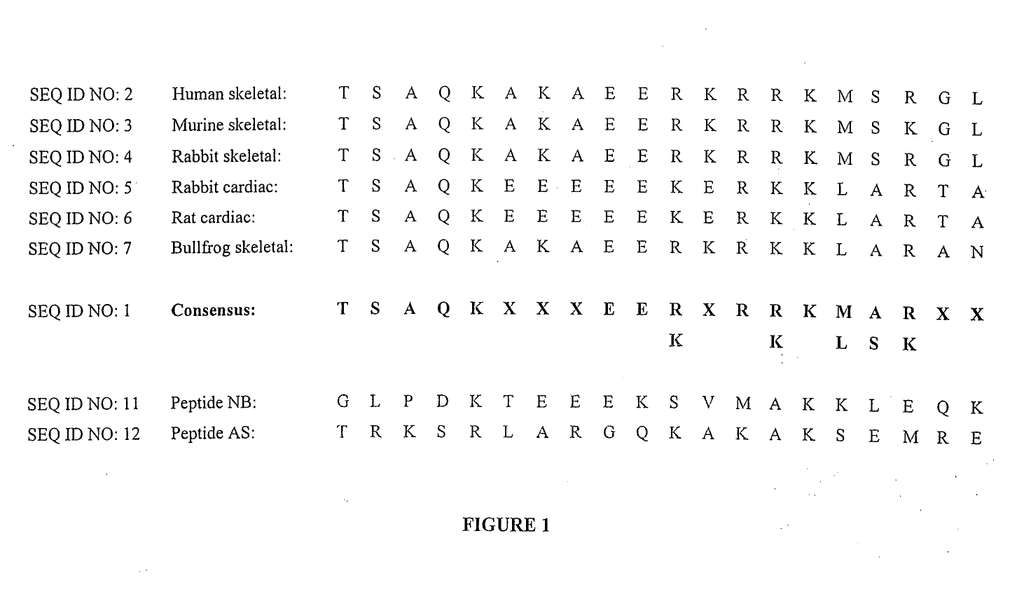

For the purposes of further describing the invention, the amino acid sequence set forth in SEQ ID NO: 1 corresponds to a 20-mer peptide consensus sequence for the cytoplasmic II-III loop of the DHPRs of skeletal and cardiac muscle of several animal species, as indicated in FIG. 1. As exemplified herein, the present inventors show that a portion of the cytoplasmic II-III loop of human skeletal muscle DHPR-3 modulates sheep RyR2 channel activity. Close sequence relationships between the various DHPRs listed in FIG. 1 indicates that any peptide having the sequence of SEQ ID NO: 1 or substantially identical to SEQ ID NO: 1 will modulate RyR2 channel activity.

Accordingly, the present invention clearly extends to the use of any and all homologues, analogues and derivatives of the amino sequence set forth in SEQ ID NO: 1. Preferably, such homologues, analogues, or derivatives will be basic charged peptides, more preferably retaining the conserved basic residues of SEQ ID NO: 1.

Even more preferably, the subject sequence comprises the motif RKRRK at amino acid positions 11 through to 15 of SEQ ID NO: 1.

In the present context, “homologues” of any one of SEQ ID NOs: 1-7 refer to those natural or synthetic basic charged peptides that are derived directly from skeletal or cardiac DHPR amino acid sequences or similar sequences from other sources, such as other special, or alternatively, by screening of mimetic peptides for RyR2 channel modulatory activity, and comprise a sequence that corresponds substantially to SEQ ID NO: 1 or any one of the specific amino acid sequences listed in SEQ ID Nos: 2-7 inclusive.

For example, amino acids of any one of SEQ ID Nos: 1-7 may be replaced by other amino acids having similar properties, for example hydrophobicity, hydrophilicity, hydrophobic moment, antigenicity, charge or propensity to form α-helical structures, provided that the overall characteristics (e.g. basic charge or conformation) of the peptide are maintained.

Substitutions encompass amino acid alterations in which an amino acid is replaced with a different naturally-occurring or a non-conventional amino acid residue. Such substitutions may be classified as “conservative”, in which case an amino acid residue is replaced with another naturally-occurring amino acid of similar character, such as, for example, Gly [symbol] Ala, Val [symbol] Ile [symbol] Leu [symbol] Met, Asp [symbol] Glu, Lys [symbol] Arg, or Asn [symbol] Gln. Amino acid substitutions are typically of single residues, but may be of multiple residues, either clustered or dispersed.

Homologues that are synthetic peptides produced by any method known to those skilled in the art, such as by solid phase methods using Fmoc amino acids in an automated peptide synthesizer, are particularly contemplated by the present invention. Such peptides may be subjected to cyclization by conventional procedures, and/or be partially purified such that they are substantially free of conspecific peptides.

“Analogues” of any one of SEQ ID NOs: 1-7, encompass those amino acid sequences that are substantially identical to said sequences or a homologue thereof, notwithstanding the occurrence of one or more non-naturally occurring amino acid analogues therein. Particularly preferred analogues include any peptide or non-peptide mimetics of any one of SEQ ID NOs: 1-7 that retain the characteristics of said sequences, such as, for example, charge distribution or conformation or other structural characteristic. The imperatoxin peptide described by Gurrola et al (1999, J. Biol. Chem. 274:7879-7886), and any synthetic non-peptide mimetics are particularly contemplated by the present invention.

The term “derivative” in relation to any one of SEQ ID NOs: 1-7 shall be taken to refer to any parts, fragments or polypeptide fusions of said sequence or a homologue or analogue thereof. Derivatives include modified amino acid sequences or peptides in which ligands are attached to one or more of the amino acid residues contained therein, such as, for example, a lipid, liposaccharide, lipopolysaccharide (LPS), carbohydrate, enzyme, peptide, radionuclide, fluorescent compound, photoactivatable residue (e.g. p-benzoylphenylalanine), or glucosyl moiety. Procedures for derivatizing peptides are well-known in the art.

For example, preferred derivatives may comprise a fragment of any one of SEQ ID NOs: 1-7 or a fragment of a homologue or analogue of any one of SEQ ID NOs: 1-7. Amino acid deletions will usually be of the order of about 1-15 amino acid residues in length. Deletions are preferably made to the N-terminus or C-terminus of SEQ ID NO: 1.

Alternatively, derivatives of any one of SEQ ID NOs: 1-7 may have additional amino acid residues added to the N-terminus or the C-terminus of the peptide or a homologue or analogue thereof. Insertions will generally be sufficiently small so as to not hinder access to the RyR2 channel, such as, for example, insertions of the order of 1-4 amino acid residues.

Preferred homologues, analogues and derivatives of any one of SEQ ID NOs: 1-7 will comprise at least about 5 contiguous amino acids of any one of SEQ ID Nos: 1-7, more preferably at least about 10 contiguous amino acid residues or more preferably at least about 15-20 contiguous amino acid residues. Accordingly, such homologues, analogues and derivatives may be full-length or less than full-length sequences compared to any one of SEQ ID NOs: 2-7.

In addition to possessing functional equivalence to SEQ ID NO: 1 in so far as RyR2 channel modulatory activity is concerned, a preferred homologue, analogue or derivative will comprise an amino acid sequence having at least about 70% identity to SEQ ID NO: 1. Preferably, the percentage identity to SEQ ID NO: 1 will be at least about 80%, more preferably at least about 90% and even more preferably at least about 95% or at least about 98 or 99%. In determining whether or not two amino acid sequences fall within defined percentage identity or similarity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP program of the Computer Genetics Group, Inc., University Research Park, Madison, Wis., United States of America (Devereaux et al, 1984, Nucl. Acids Res. 12: 387-395), which utilizes the algorithm of Needleman and Wunsch (1970, J. Mol. Biol. 48: 443-453) or alternatively, the CLUSTAL W algorithm of Thompson et al (1994, Nucl. Acids Res. 22: 4673-4680) for multiple alignments, to maximize the number of identical/similar amino acids and to minimize the number and/or length of sequence gaps in the alignment.

Particularly preferred homologues, analogues, or derivatives of SEQ ID NO: 1 will successfully compete with any one of SEQ ID NOs: 2-7 for modulation of cardiac RyR2 channel activity. In standard competition studies, different concentrations of the peptide being tested are assayed for RyR2 channel modulatory activity in the presence of concentrations of SEQ ID NO: 2, for example, that either enhance or inhibit cardiac RyR2 channel activity. For example, a high affinity activator of a cardiac RyR2 channel can be determined by its ability to successfully enhance channel activity in a lipid bilayer at a cytoplasmic calcium concentration in the range of about 10⁻⁷M to about 10⁻⁵M, and to compete for the enhancement of channel activity that is induced by known activator peptide (e.g. about 1 nM SEQ ID NO: 2 to about 100 nM SEQ ID NO: 2). Similarly, a high affinity inhibitor of a cardiac RyR2 channel can be determined by its ability to successfully inhibit channel activity in a lipid bilayer at a cytoplasmic calcium concentration in the range of about 10⁻⁷M to about 10⁻⁵M, and to compete for the inhibition of channel activity that is induced by known a inhibitor peptide at cytoplasmic Ca²⁺ concentrations up to 10⁻⁴N (e.g. 32.5 μM SEQ ID NO: 2).

Without limiting the present invention in any way, the inventors have generated three peptides which correspond to analogues and derivatives, as defined herein, of the human skeletal DHPR 20-mer peptide sequence described in SEQ ID NO: 2. These peptides are detailed below:

(i) SEQ ID NO: 8 corresponds to the 20-mer peptide of SEQ ID NO: 2 but wherein serine⁶⁸⁷ (residue 17 of SEQ ID NO: 2) is replaced by an alanine residue, as follows:

| | |
|---|---|
| TSAQKAKAEERKRRKMARGL | (SEQ ID NO: 8) |

(ii) SEQ ID NO: 9 corresponds to the 20-mer peptide of SEQ ID NO: 2 but wherein arginine⁶⁸⁸ (residue 18 of SEQ ID NO: 2) is replaced by the D isomer of arginine, as follows:

| | |
|---|---|
| TSAQKAKAEERKRRKMSR_{D}GL | (SEQ ID NO: 9) |

(iii) SEQ ID NO: 10 corresponds to the 20-mer peptide of SEQ ID NO: 2 but wherein both serine⁶⁸⁷ (residue 17 of SEQ ID NO: 2) is replaced by an alanine residue and arginine⁶⁸⁸ (residue 18 of SEQ ID NO: 2) is replaced by the D isomer of arginine, as follows:

| | |
|---|---|
| TSAQKAKAEERKRRKMAR_{D}GL | (SEQ ID NO: 10) |

In the present context, for example, the term “contacting” may mean a cardiac RyR2 channel with a basic peptide fragment of a DHPR polypeptide is meant that the peptide is brought into close physical association with the channel without necessarily requiring actual binding of the peptide to the channel. Notwithstanding that the DHPR peptide may bind to the RyR2 channel in performing the invention, such binding is not an essential feature of the invention, because all that is required is modified channel activity. In this respect, the purpose of the invention is not to achieve binding to the cardiac RyR2 polypeptide, but to modulate activity of the RyR2 channel. Those skilled in the art are aware that binding and activity are not necessarily equivalent, because a DHPR peptide may bind to any one of a multitude of different sites on a RyR2 polypeptide without necessarily modifying the activity of the RyR2 channel.

Preferably, the peptide is contacted with the cytoplasmic face of the RyR2 channel.

Preferably, any one of SEQ ID NOs: 1-7 or a homologue, analogue, or derivative of any one of SEQ ID NOs: 1-7, such as any one of SEQ ID NOS: 8-10, binds to a part of the RyR2 polypeptide in the RyR2 channel during performance of the inventive method. Without being bound by any theory or mode of action, the peptide (or a homologue, analogue, or derivative thereof) used in performing the invention can assume a conformation that gains access to the RyR2 channel by binding to one or more negatively charged residues of the RyR2 polypeptide in the channel, such as, for example, acidic residues in the amino acid sequence: FRAEKTYAVKAGRWYFEFEAVTSGDMRVGWSRPGCQP (SEQ ID NO: 13).

It follows from the preceding discussion that, to determine activity of the cardiac RyR2 calcium channel, it is not sufficient to merely measure binding of the peptide, or ryanodine for that matter, to the channel or to the RyR2 polypeptide, although that may certainly form an adjunct to determining modified channel activity. Preferred means for determining channel activity are selected from the group consisting of:
(i) recording of single or multiple RyR2 channel openings in lipid bilayers using art-recognized procedures, such as, for example, those described by Ahern et al. (1994, FEBS Lett. 352:369-374), Lu et al., (1994, J. Biol. Chem. 269:6511-6516), Layer et al. (1995, J. Membr. Biol. 147:7-22), or Dulhunty et al. (1999) supra;
(ii) determination of calcium release from SR vesicles using art-recognized procedures, such as, for example, those described by El-Hayek et al. (1995, supra), Gurrola et al. (I 999, supra), or Dulhunty et al. (1999, supra);
(iii) determination of cardiac function, such as, for example, by determining cardiac contractility according to Zaloga et al., (1997); and
(iv) determination of vascular tone of isolated thoracic aortic rings following peptide administration using any art recognized method for determining vascular tone.

Those skilled in the art are aware that cardiac function can be readily determined by measuring the concentration dependence of peptide administration on one or more parameters selected from the group consisting of contractility as assessed by dP/dtmax or −dP/dtmax values, left ventricular systolic pressure, and heart rate. Ventricular fibrillation or other cardiac arrhythmia can also be determined to quantify negative side-effects of peptide.

In assaying for enhanced cardiac RyR2 channel activity, an enhanced channel opening probability (Po) is detected. The higher channel opening probability leads to enhanced calcium efflux or release from the SR, including from SR vesicles, and enhanced cardiac contractility, and reduced relaxation. Vascular tone may also be enhanced.

In contrast, inhibitors of cardiac RyR2 channel activity will decrease the channel open probability, reduce calcium release from the SR, and reduce cardiac contractility. Vascular tone may also be reduced.

Additional methods of determining modified cardiac RyR2 channel activity are not excluded.

The efficacy of any one of SEQ ID NOs: 1-7 or a homologue, analogue or derivative thereof (such as SEQ ID NOs: 8-10), in particular SEQ ID NO: 2, in modulating cardiac RYR channel activity, establishes the utility of such sequences as a reagents for use in screening for compounds sharing structural and functional similarity. Such a reagent enables a high-throughput screening assay in which thousands of compounds can be rapidly screened. Peptide mimetics potentially having the modulatory activity described herein can be tested for their ability to modulate cardiac RyR2 channel activity with isolated cardiac RyR2 receptors in lipid bilayers, or in another suitable assay format.

Accordingly, a second aspect of the invention provides a method for identifying a peptide or non-peptide modulator of a cardiac RyR2 calcium channel comprising:
(i) incubating an amount of a fragment of a dihydropyridine receptor polypeptide or a homologue, analogue or derivative thereof that modulates cardiac RyR2 channel activity in the presence of a functional cardiac RyR2 calcium channel under conditions appropriate for calcium channel activity to be modulated and determining the activity of the channel;
(ii) incubating a candidate peptide or non-peptide compound in the presence of said functional cardiac RyR2 calcium channel under conditions appropriate for calcium channel activity to be modulated by said dihydropyridine receptor polypeptide or a homologue, analogue or derivative thereof and determining the activity of the channel; and
(iii) comparing the activity at (i) and (ii).

Preferably said fragment comprises at least 5 contiguous amino acids of the peptide sequence of SEQ ID NO: 1.

Preferably, those peptides having comparable or enhanced modulation of channel activity to SEQ ID NO: 1 or a homologue, analogue or derivative of SEQ ID NO: 1 are selected. Such peptides are detectable by the comparable or enhanced modulation of channel activity detected at (ii) relative to (i) supra.

For example, a fixed amount of SEQ ID NO: 1 or a homologue, analogue or derivative thereof which modulates cardiac RyR2 channel activity can be added to the functional receptor. The reaction mixture is then incubated under conditions appropriate for activity to be modulated, and activity of the channel is determined. In a parallel experiment, a candidate peptide or non-peptide compound is incubated with the cardiac channel under conditions that permit modulation of activity in the presence of SEQ ID NO: 1 and the channel activity of this test sample is determined relative the activity of SEQ ID NO: 1 or its homologue, analogue or derivative. Those peptides or non-peptide compounds having comparable or enhanced modulatory activity relative to SEQ ID NO: 1 or its homologue, analogue or derivative, can be selected.

In an alternative embodiment, this aspect of the invention provides a method for identifying a peptide or non-peptide modulator of a cardiac RyR2 calcium channel comprising:
(i) incubating an amount of a fragment of a dihydropyridine receptor polypeptide or a homologue, analogue or derivative thereof that modulates cardiac RyR2 channel activity in the presence of a functional cardiac RyR2 calcium channel under conditions appropriate for calcium channel activity to be modulated and determining the activity of the channel;
(ii) incubating a candidate peptide or non-peptide compound and an amount of said dihydropyridine receptor polypeptide or a homologue, analogue or derivative thereof that modulates cardiac RyR2 channel activity in the presence of a functional cardiac RyR2 calcium channel under conditions appropriate for calcium channel activity to be modulated by said dihydropyridine receptor polypeptide or a homologue, analogue or derivative thereof and determining the activity of the channel; and
(iii) comparing the activity at (i) and (ii).

Preferably, said fragment comprises at least 5 contiguous amino acids of the peptide sequence of SEQ ID NO: 1.

In a most preferred embodiment of these aspects of the present invention, said SEQ ID NO: 1 peptide or homologue or derivative thereof is selected from any one or more of SEQ ID NOs: 2-10.

Preferably, those peptides or non-peptide mimetics etc having comparable or enhanced modulation of channel activity to SEQ ID NO: 1 or a homologue, analogue or derivative of SEQ ID NO: 1 are selected. Such peptides or non-peptide compounds are detectable by the comparable or enhanced modulation of channel activity detected at (ii) relative to (i) supra.

For example, a fixed amount of SEQ ID NO: 1 or a homologue, analogue or derivative of SEQ ID NO: 1 that modulates cardiac RyR2 channel activity is added to the functional receptor under conditions that permit modulation to occur. The activity of the channel is determined in the presence or absence of a candidate peptide and the channel activities of the samples compared. Those peptides or non-peptide compounds that modulate the effect of SEQ ID NO: 1, or its homologue, analogue or derivative, on cardiac RyR2 calcium channel activity can be selected.

Any conventional assay format which relies on modulation of cardiac RyR2 channel activity is appropriate for this purpose. In a preferred assay format, the sample to be tested is a reconstituted RyR2 channel in a planar lipid bilayer or a SR vesicle or a dyspedic myocyte having a functional cardiac RyR2 calcium channel. Using only routine experimentation, one skilled in the art can determine whether a particular candidate peptide, or non-peptide compound, modulates activity of a cardiac calcium channel.

The present invention clearly contemplates a process that utilizes rapid, high throughput screens with some tolerance of non-specificity and/or smaller-scale functional screens having higher specificity, and/or quantitative kinetic studies to elucidate chemical structure/function relationships of the cardiac RyR2 channel, such as, for example, the determination of peptide or non-peptide compounds that are agonists or antagonists of cardiac RyR2 calcium channel function, or the elucidation of the docking site(s) for said compounds in the channel.

Preferably, the present invention contemplates a process comprising:
(i) identifying candidate agonists and antagonists of a cardiac RyR2 calcium channel;
(ii) determining those compounds at (i) that actually activate or inhibit the activity of a cardiac RyR2 channel;
(iii) determining which compounds at (ii) have higher binding affinities for said cardiac RyR2 calcium channel than any one of SEQ ID Nos:1-10; and
(iv) optionally, determining the sites of interaction between those compounds at (iii) and said cardiac RyR2 calcium channel.

Rapid, high throughput screens to identify candidate agonists and antagonists of a cardiac RyR2 calcium channel are preferably carried out using cardiac RyR2 calcium channels in microsomal preparations of cardiac muscle, or alternatively, expressed in CHO cells or other suitable cell-based assay system. Such high throughput screens facilitate the screening of large numbers of compounds incubated with the microsomal preparations, or injected into or incubated with transfected cells expressing the RyR2 channel. High throughput screens also facilitate the screening of large numbers of peptides expressed from libraries that have been transfected into cells expressing the RyR2 channel.

Alternatively, or in addition, candidate agonist and antagonist molecules are identified by binding the cardiac RyR2 protein to a support such as a plurality of polymeric pins and bringing the polypeptide on the plurality of pins into contact with candidate agonist and/or antagonist molecules for screening. The molecules being screened may be isotopically labelled so as to permit ready detection of binding. Alternatively, compounds for screening may be bound to a solid support, such as a plurality of pins which are reacted with the RyR2 polypeptide. Binding may, for example, be determined again by isotopic-labelling, or by antibody detection, or use of another reporting agent.

The binding affinity of a particular chemical compound for a cardiac RyR2 calcium channel may be determined by any assay known to those skilled in the art to be useful for determining kinetic parameters of protein-ligand interactions. Preferably, a binding assay, such as, for example, surface plasmon resonance, is employed. The surface plasmon resonance of a protein may be determined, for example, using a Biacore™ analyzer. As will be known to those skilled in the art, this method provides data for the determination of on and off rates for the binding of a ligand to a protein of interest.

To screen multiple candidate compounds, the compounds may be attached to a plurality of polymeric pins or supports.

To determine the site(s) of interaction between a candidate compound and the cardiac RyR2 calcium channel, the binding of the candidate compounds to various mutant RyR2 polypeptides having one or more sites in the native protein deleted or substituted with a variant amino acid sequence can be determined, and compared to the binding of said compound to the wild-type or non-mutant RyR2 protein. Again, surface plasmon resonance may be employed to facilitate the comparison of binding affinities. Data on those sites of interaction which provide stronger agonist/antagonist activity are used to facilitate the rational design of drug which bind to such sites, albeit with enhanced binding affinities.

Compounds detected using this screening procedure can ultimately be used, for example, in the treatment of cardiac dysfunction.

In an alternative embodiment, agonist and/or antagonist compounds are identified using rational drug design; by identifying compounds which bind to or associate with the three-dimensional structure of the cardiac RyR2 calcium channel. The present invention clearly contemplates any synthetic compounds derived from the three dimensional structure of the cardiac RyR2 calcium channel that bind to said channel and that activate or inhibit cardiac RyR2 calcium channel activity.

The observation that the open probability of a cardiac RyR2 channel is modified by incubation with SEQ ID NO: 1 or a homologue, analogue or derivative thereof establishes the utility of such sequences in establishing or determining the pore structure and regulation mechanism of the cardiac RyR2 channel in vitro and in vivo. More particularly, the correlation between binding of the peptide to RyR2 and high open probability of the channel on the one hand, and the correlation between non-specific binding of peptide at negatively charged residues within the channel pore and low open probability, makes possible the prediction of open probability based upon peptide binding studies.

Accordingly, a third aspect of the present invention provides a method of determining whether a cardiac RyR2 channel is open or has a high channel open probability said method comprising contacting a cardiac RyR2 channel with an amount of a fragment of a dihydropyridine receptor (DHPR) polypeptide or homologue, analogue or derivative thereof for a time and under conditions sufficient for binding to RyR2 to occur and determining the binding of said peptide to RyR2, wherein binding of said peptide to RyR2 indicates a high channel open probability and wherein non-specific peptide binding of peptide to the channel pore indicates a low channel open probability.

Preferably, said fragment is a peptide substantially as defined in any one or more of SEQ ID NOs: 1-10.

Peptide binding can be determined by any method known to the skilled person, such as, for example, by using a radioactive labeled or fluorescent labeled peptide, or peptide labeled with a reporter molecule, and determining the amount of label or reporter molecule bound to the channel at any particular concentration of peptide. Preferred reporter molecules for this purpose are small molecules that do not hinder the ability of the peptide to bind to the channel, such as, for example, photoactivatable compounds.

Alternatively, peptide binding may be determined indirectly by measuring the activity of the channel, of the calcium release through the channel. As exemplified herein, peptide at a concentration between about 1 nM and about 10 μM peptide produces a high channel open probability, whereas peptide at a higher concentration indicates a low channel open probability, particular for channels in lipid bilayers.

A fourth aspect of the present invention provides a method of treatment of cardiac dysfunction in a human or animal subject comprising administering an effective amount of a fragment of a dihydropyridine receptor (DHPR) polypeptide or derivative, homologue or analogue thereof for a time and under conditions sufficient for enhanced cardiac contraction to occur thereby rectifying said cardiac dysfunction.

Preferably, said fragment comprises at least 5 contiguous amino acids of a peptide substantially as defined in any one or more of SEQ ID NOs: 1-10.

By “cardiac dysfunction” is meant a condition involving impaired myocardial contraction, such as, for example, wherein Ca²⁺ sensitivity of the myofilaments is reduced, or there is a deterioration of calcium signalling, such as, for example, by deterioration or disruption of the calcium synapse, deterioration of the RyR2, or deterioration of the DHPR. Conditions of cardiac dysfunction contemplated herein to be amenable to treatment according to the present invention include, but are not limited to, myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug (e.g. cocaine)-induced cardiomyopathy, infarction, dysrhythmia, congestive heart failure, or heart attack.

By “effective amount” means an amount of a peptide sufficient to diminish or reverse progression of the dysfunction.

For purposes of this aspect of the invention, beneficial or desired clinical results include, but are not limited to, alleviation of symptoms, diminishment of extent of disease, stabilization of the disease state, delay or slowing of disease progression, amelioration or palliation of the disease state, and remission (whether partial or total), whether detectable or undetectable. “Treatment” also includes prolonging survival as compared to the expected survival of a subject not receiving treatment. As used herein, the term “treatment” includes prophylaxis.

“Palliating” a disease means that the extent and/or undesirable clinical manifestations of a disease state are lessened and/or the time course of the progression is slowed or lengthened, by treatment.

In the context of prophylaxis, a “subject” includes, but is not limited to, individuals in the general population who are about 40 years of age or older; and, in particular, an individual with a history or predisposition to developing cardiac hypertrophy, cardiac myopathy, heart attack, hypertension, renal failure, vascular hypertension, respiratory ailment such as emphysema or cystic fibrosis, chronic asthma, and tuberculosis. Suitable subjects also include organ transplant patients.

In a fifth aspect of the present invention there is provided the use of a fragment of a dihydropyridine receptor polypeptide comprising at least 5 contiguous amino acid residues of the peptide set forth in any one of SEQ ID NOs: 1-10 or a homologue, analogue or derivative thereof to modify the activity of a cardiac ryanodine calcium channel, thereby modifying defective calcium signaling.

Preferably, said defective calcium signaling induces chronic hypertrophy, dilated cardiac myopathy or heart failure.

In a sixth aspect of the present invention there is provided the use of a fragment of a dihydropyridine receptor polypeptide comprising at least 5 contiguous amino acid residues of the peptide set forth in any one of SEQ ID NOs: 1-10 or a homologue, analogue or derivative thereof in the manufacture of a medicament for the treatment of cardiac dysfunction in a human or animal subject. This invention involves the use of a peptide set forth in any one of SEQ ID Nos: 1-10 or a homologue, analogue, or derivative thereof, to modify the activity of cardiac RyR2 calcium channel, thereby modifying defective calcium signaling that induces chronic hypertrophy or dilated cardiac myopathy or even heart failure in the chronic untreated animal of human subject.

Preferably, the peptide or homologue, analogue or derivative thereof is administered at a dosage that can enhance contractile force, and further increase intracellular calcium concentration (i.e. [Ca²⁺]ᵢ) during systole, and further decrease [Ca²⁺]ᵢ during diastole. Preferably, the peptide or a homologue, analogue or derivative thereof induces at least about a 3% or 5% increase in systolic [Ca²⁺]ᵢ relative to the systolic [Ca²⁺]ᵢ determined in the absence of the peptide from a standard in vitro calcium-sensitizing assay. Preferably, the peptide or a homologue, analogue or derivative thereof induces at least about a 3% or 5% decrease in diastolic [Ca²⁺]ᵢ relative to the diastolic [Ca²⁺]ᵢ determined in the absence of the peptide from a standard in vitro calcium-sensitizing assay. More preferably, systolic [Ca²⁺]ᵢ is increased, or diastolic [Ca²⁺]ᵢ is decreased, by at least about 10% or 15%, and still more preferably by at least about 20%, 25%, 30%, 40% or 50%, relative to the systolic [Ca²⁺]ᵢ or diastolic [Ca²⁺]ᵢ respectively, that is measured in absence of the peptide in such a standard in vitro calcium-sensitizing assay.

Even more preferably, the administered peptide, or a homologue, analogue or derivative thereof, improves the efficiency of cardiac contraction. Preferably, cardiac contraction is enhanced by inducing at least about a 5% or 10% increase in preload-recruitable stroke work (PRSW) at within 0.5-1.0 hr following administration. More preferably, cardiac contraction is enhanced by about 15%, 20%, 30%, 40%, 50%, 55%, 60% or 70% as determined by an increase in PRSW in heart failure subjects relative to healthy individuals.

For example, the peptide, or a homologue, analogue or derivative thereof, can be immediately administered to a patient (e.g. i.p. or i.v.) that has suffered or is suffering from congestive heart failure or cardiogenic shock. Such immediate administration preferably would entail administration of a suitable dosage of peptide to enhance RyR2 calcium channel activity within about 1, 2, 4, 8, 12 or 24 hours, or from more than one day to about 2 or three weeks, after the subject has suffered from heart failure such as congestive heart failure or cardiogenic shock.

Relatively long-term administration of the peptide, or a homologue, analogue or derivative thereof, at a dosage suitable to activate RyR2 calcium channel activity will also be beneficial after a patient has suffered from chronic heart failure, to provide increased exercise tolerance and functional capacity. For example, the peptide can be administered regularly to a patient for at least 2, 4, 6, 8, 12, 16, 18, 20 or 24 weeks, or longer such as, for example, 6 months, 1 years, 2 years, 3 years or more, after having suffered heart failure to promote enhanced functional capacity. An oral dosage formulation would be preferred for such long-term administration.

The administration of the peptide, or a homologue, analogue or derivative thereof, at a dosage that inhibits cardiac RyR2 calcium channel activity in human or animal subjects is not excluded, and may be appropriate, for example, in cases where transient relaxation of cardiac tissue is required.

Toxicity, and the therapeutic efficacy of the peptide, homologue, analogue or derivative, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and this index can be expressed as the ratio LD₅₀/ED₅₀. The amino acid sequence set forth in any one of SEQ ID NOs: 1-7, or a homologue, analogue, or derivative thereof having a high therapeutic index, is preferred.

Whilst peptides, homologues, analogues, or derivatives, that exhibit toxic side effects or have a high LD₅₀ value are less desirable, such peptides may be used in conjunction with a delivery system that targets such compounds to the site of affected tissue, thereby minimizing potential damage to healthy tissue.

Data obtained from cell based assays and animal studies can be used in formulating a range of dosage of the subject peptides for use in humans. The animal models of cardiac hypertrophy described by Grant et al. (U.S. Pat. No. 6,201,165 issued Mar. 13, 2001) are particularly useful for this purpose. The dosage of peptide, homologue, analogue, or derivative, lies preferably within a range of concentrations that, following administration by a particular route, produce a circulating concentration consistent with the ED₅₀ and having little or no toxicity. The dosage may vary within this range depending upon the dosage form and route of administration. The dosage may also vary according to factors such as the disease, severity of disease, age, sex, and weight of the individual.

For any peptide, homologue, analogue, or derivative, used in the method of the invention, the therapeutically effective dose can be estimated initially from cell based assays or animal models. For example, an effective dose may be formulated in animal models to achieve a circulating plasma concentration range that includes the IC₅₀ (i.e., the concentration of peptide or non-peptide compound that achieves a half-maximal inhibition of symptoms) determined from cell based assays and/or in whole animals. Such information can be used to more accurately determine useful doses in humans.

Suitable dosages of the peptide, or a homologue, analogue or derivative thereof, can also be determined by inducing heart failure in an animal model by chronic rapid ventricular pacing, an then infusing different concentrations of the peptide into the right atrium at a rate of about 3.3 mL/min and then recording the pressure-dimension relationships and the arterial pressure response to the peptide. Control experiments using known calcium sensitizers, such as those described by Marban (U.S. Pat. No. 6,191,136 issued on Feb. 20, 2001) can also be performed. Cardiac oxygen consumption also may be measured.

Yet another aspect of the present invention is directed to a pharmaceutical composition comprising a fragment of a dihydropylidine receptor polypeptide, which peptide comprises at least about 5 contiguous amino acid residues of the peptide set forth in any one of SEQ ID NOs: 1-10, or a homologue, analogue or derivative thereof together with one or more pharmaceutically acceptable carriers and/or diluents.

The therapeutic efficacy of a substance detected by the methods of the present invention in the treatment of cardiac dysfunction can be accomplished by those skilled in the art using known principles of diagnosis and treatment.

Therapeutic compositions must be sterile and stable under the conditions of manufacture and storage. The peptides can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Enhanced absorption can be achieved by conjugation of the peptide, or a homologue, analogue or derivative thereof, to a lipid or liposaccharide moiety.

Prolonged absorption of the injectable compositions can be brought about by including in the composition an agent that delays absorption, for example, a monostearate salt or gelatin. Moreover, the compounds can be administered in a time release formulation, for example in a composition which includes a slow release or controlled release polymer, preferably comprising a hydrophobic and/or amphiphilic compound. The peptides can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Methods for the preparation of such formulations are generally known to those skilled in the art.

For example, a suitable controlled-release delivery ampoule can be prepared by dispersing the peptide, homologue, analogue or derivative, in a bioerodable, biodegradable poly(ε-caprolactone) polymer matrix in the melt stage, provided the peptide or protein drug is dispersed in a glassy matrix phase having a glass transition temperature that is higher than the melting point of the poly(ε-caprolactone) polymer, such as, for example, a glassy matrix phase produced by lyophilizing an aqueous solution of the peptide and a suitable thermoprotectant (e.g. trehalose, melezitose, lactose, maltose, cellobiose, melibiose, or raffinose) as described by Wang et al. (U.S. Pat. No. 6,187,330).

Sterile injectable solutions can be prepared by incorporating the peptide, homologue, analogue or derivative, in the required amount in an appropriate solvent with one or a combination of ingredients enumerated above, as required, followed by filtered sterilization. Generally, dispersions are prepared by incorporating the active peptide or non-peptide compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Preferably, the peptide of the invention, or a homologue, analogue or derivative thereof, is formulated to extend its half-life following administration, particularly in formulations for the treatment of chronic conditions. Methods for extending the half-life of the active peptide compound includes direct modification to reduce its proteolysis, such as, for example, by cross-linking or amino acid substitutions to remove sites for known proteases. Alternatively, half-life of any active ingredient can be extended by encapsulating it in a suitable formulation, such as, for example, a slow release formulation. Depending on the route of administration, the peptide, homologue, analogue or derivative, may be coated in a material to protect it from the action of enzymes, acids and other natural conditions which may lead to its inactivation.

For example, the peptide, homologue, analogue or derivative, can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes.

Dispersions can also be prepared in glycerol, liquid polyethylene glycols, and mixtures thereof and in oils. Under ordinary conditions of storage and use, these preparations may contain a preservative to prevent the growth of microorganisms.

This invention is also described with reference to the following non-limiting examples.

### EXAMPLE 1

### A 20-Mer Peptide That Modulates Cardiac RyR2 Calcium Channel Activity

### Materials & Methods

Materials.

Chemicals and biochemicals were from Sigma-Aldrich (Castle Hill, Australia). The DHPR II-III loop peptides (SEQ ID Nos: 1-7) were synthesized using an Applied Biosystems 430A Peptide Synthesizer with purification to ≧98% using HPLC and mass spectroscopy and NMR. Peptide was prepared in a ˜2 mM stock solution in H₂O and frozen in 20 μl aliquots. Precise stock solution concentrations were determined by Auspep Pty Ltd using acid hydrolysis followed by a standardized PTC (phenylthiocarbamyl) protocol and analysed by reverse phase HPLC.

Peptides

Particular peptides used for the study were:

1. The 20-mer peptide of the II-II cytoplasmic loop of DHPR (SEQ ID NO: 2):

Thr Ser Ala Gln Lys Ala Lys Ala Glu Glu Arg Lys
Arg Arg Lys Met Ser Arg Gly Leu

2. Peptide NB (the N-terminal portion of the B segment of the II-III loop; Hamilton and Ianuzzo, 1991; SEQ ID NO: 11):

Gly Leu Pro Asp Lys Thr Glu Glu Glu Lys Ser Val
Met Ala Lys Lys Leu Glu Gln Lys

3. Peptide A1S (scrambled 20-mer peptide; SEQ ID NO: 12):

Thr Arg Lys Ser Arg Leu Ala Arg Gly Gln Lys Ala
Lys Ala Lys Ser Glu Met Arg Glu

SR Vesicle Preparation.

SR vesicles were isolated from sheep heart as described by Layer et al. (1995, J. Membr. Biol. 147:7-22).

Lipid Bilayers.

Experiments were carried out at 20° C. to 25° C., essentially according to Ahern et al. (1994, FEBS Lett. 352:369-374) and Layer et al. (1995, J. Membr. Biol. 147:7-22). Bilayers were formed from phosphatidylethanolamine, phosphatidylserine and phosphatidylcholine (5:3:2 w/w/w) (Avanti Polar Lipids, Alabaster, Ala.) across an aperture with a diameter of ˜00 μm in the wall of a 1.0 ml Delrin cup (Cadillac Plastics, Australia). Terminal cisternae (TC) vesicles (final concentration, 10 μg/ml) and drugs were added to the cytoplasmic (i.e. cis) chamber. The bilayer potential was controlled, and single channel activity recorded, using an Axopatch 200A amplifier (Axon Instruments, Foster City, Calif.). For experimental purposes, the cis chamber was held at ground and the voltage of the lumen (i.e. trans) chamber controlled. Bilayer potential is expressed in the conventional way as V_{cis}−Vₜᵣₐₙₛ, (i.e. V_{cytoplasm}−Vₗᵤₘₑₙ).

Bilayer Solutions.

Bilayers were formed as described above and vesicles were incorporated into the bilayer using a cis solution containing 230 mM Cs methanesulphonate (MS), 20 mM CsCl, 1 mM CaCl₂ and 10 mM N-tris[hydroxymethyl]methyl-2-aminoethanesulfonic acid (TES, pH 7.4 with CsOH), and a trans solution containing 30 mM Cs MS, 20 mM CsCl, 1 mM CaCl₂ and 10 mM TES (pH7.4).

To prevent incorporation of multiple channels into the bilayer, the cis solution was replaced by perfusion of the cis chamber when channel activity was observed. The cis perfusion solution was identical to the initial cis solution, except that the [Ca²⁺] was 3×10⁻⁷M, buffered using 1 mM BAPTA. CsMS (200 mM) was added to the trans chamber following channel incorporation to produce symmetrical solutions.

Recording Single Channel Activity and Data Analyses.

Bilayer potential was changed every 30 s, over 2 min following each addition of peptide to the cis chamber. Control activity was recorded for 2 min after addition of 200 mM CsMS to the trans chamber. Activity was then recorded for 2 min after cis addition of each aliquot of peptide (six different peptide concentrations were examined in each channel). Peptides were perfused out of the cis chamber, and the recovery recorded. Channels were finally exposed to 30 μM ruthenium red.

Currents were filtered at 1 kHz (8-pole low pass Bessel, −3 dB) and digitized at 5 kHz. Analysis of single channel records (using Channel 2, developed by P W Gage and M Smith) yielded channel open probability (Pₒ), frequency of events (Fₒ), open times, closed times and mean open or closed times (Tₒ or T_{c}) as well as mean current (I′). The event discriminator was set above the baseline noise at ˜20% of the maximum current, rather than the usual 50%, and so openings to both sub conductance and maximum conductance levels were included in the analysis. Channel activity was analyzed over two 30 s periods of continuous activity at +40 mV and two 30 s periods of continuous activity at −40 mV.

Statistical Analyses.

Average data is given as mean±SEM. The significance of the difference between control and test values was tested using one sided or two sided Students T-tests, for independent or paired data, as appropriate. Differences were considered to be significant when P≦0.05.

### EXAMPLE 2

### A 20-Mer Peptide that Modulates Cardiac RyR2 Calcium Channel Activity

### Results

An increase in the activity of RyRs from cardiac muscle was seen when the 20-mer peptide (SEQ ID NO: 2) was added to the cytoplasmic (cis) side of the channel at a concentration of 10⁻⁷ cis Ca²⁺. Single channels were identified as RyRs by their Cs⁺ conductance of ˜450 pS with bilayer potentials of +40 mV or −40 mV and by the ability of 30 μM ruthenium red to block the channel at the end of the experiment.

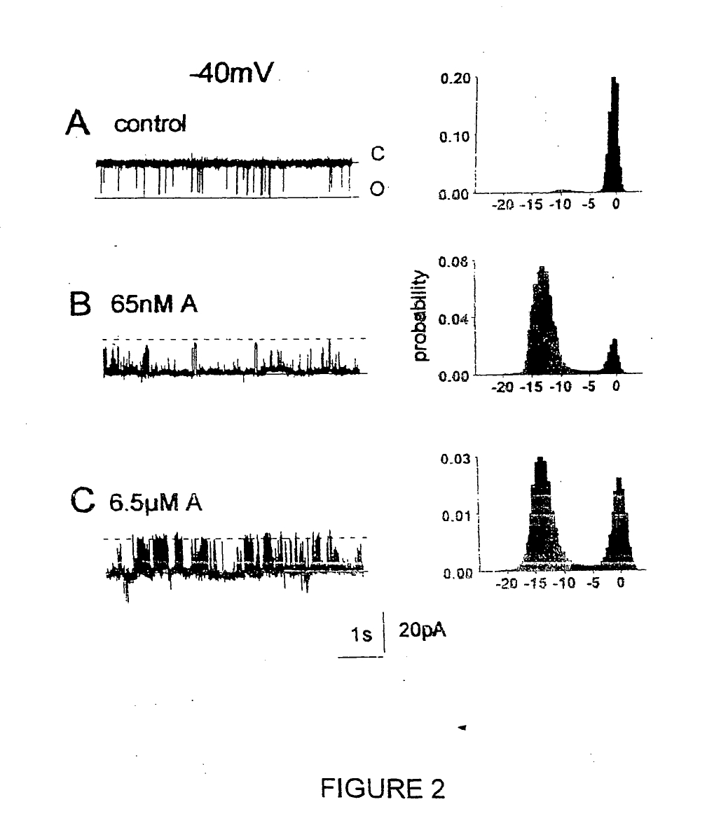

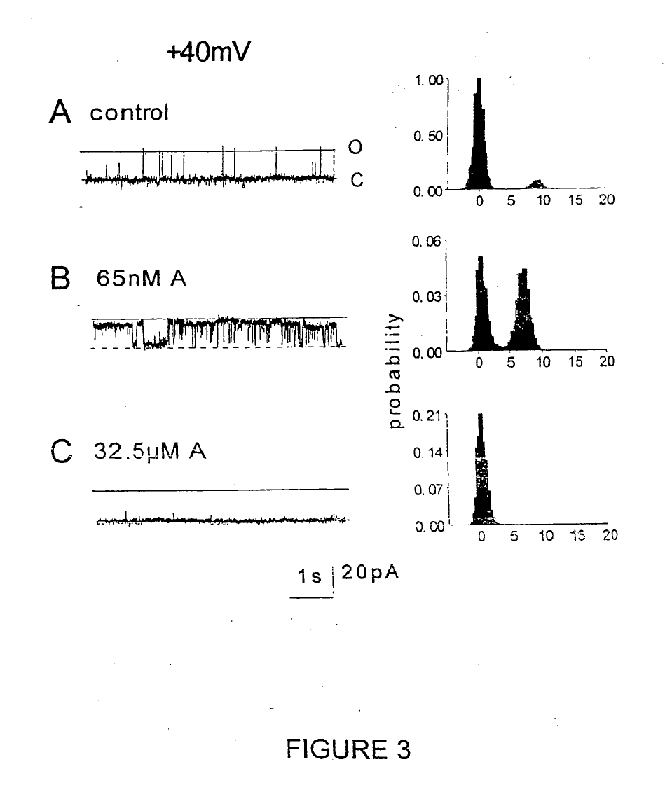

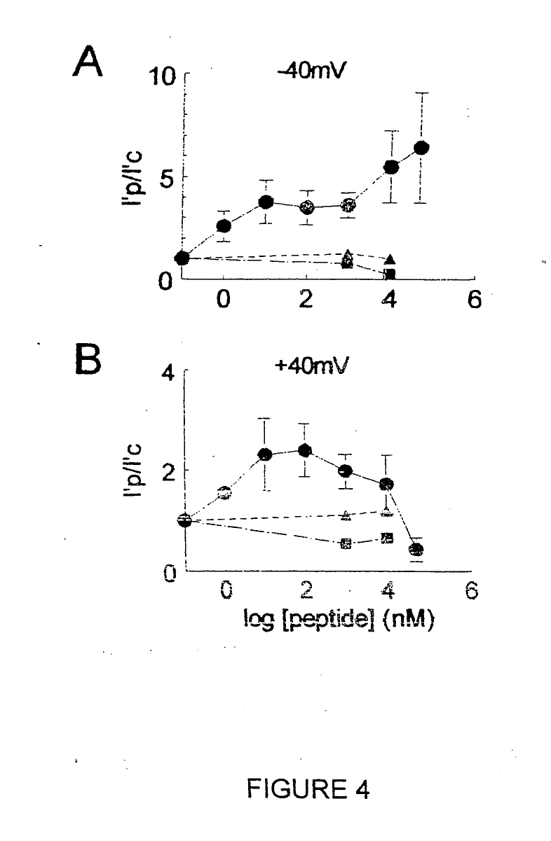

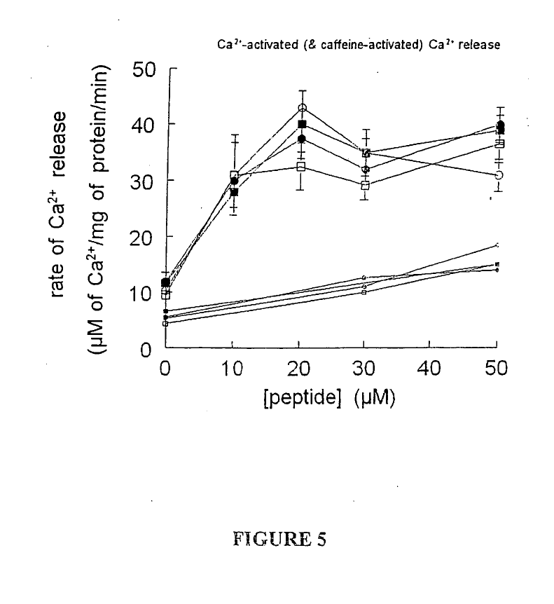

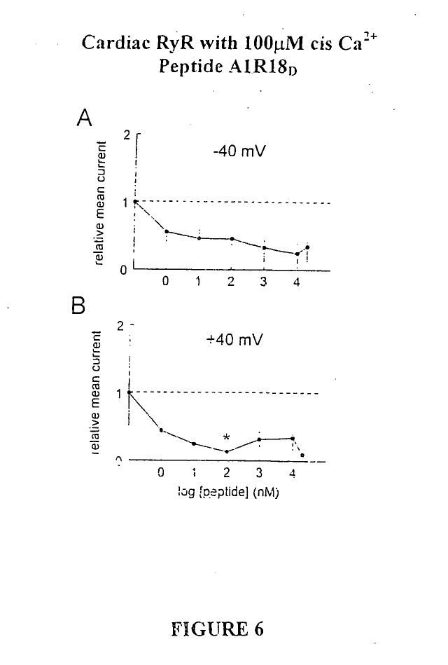

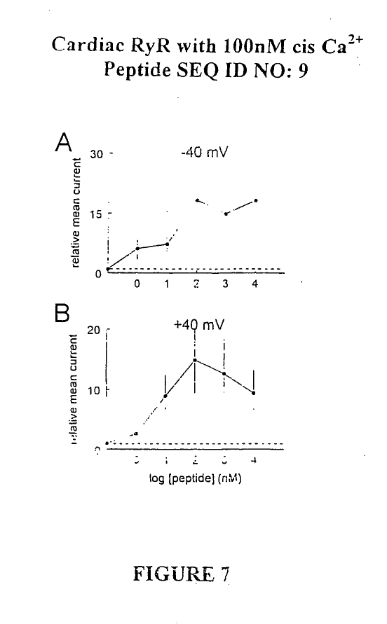

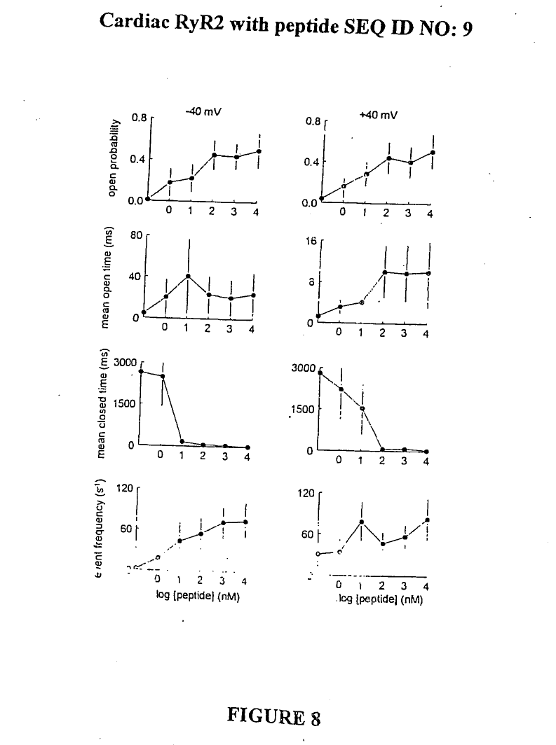

Records from one experiment in which cardiac RyRs were strongly activated by the 20-mer peptide (SEQ ID NO: 2) are shown in FIG. 2, at −40 mV, and in FIG. 3, at +40 mV. Channel activity before addition of the peptide consisted of brief intermittent openings (FIG. 2, panel A; FIG. 3, panel A). Within 10 s of adding 65 nM peptide (SEQ ID NO: 2), channel openings increased at −40 mV.

An increase in the frequency of events, and the appearance of very long openings, were accompanied by the opening of a second channel in the bilayer (FIG. 2, panel B). RyR2 channel activity at −40 mV fell slightly when peptide concentration was increased to 6.5 μM SEQ ID NO: 2, although the activity of the second channel was more pronounced. As shown in FIG. 2, panel C, the channel shows a high open probability (Po), particularly at 65 nM peptide (the predominant current level, O, expected if the channel was fully open). a further 4-fold in 50 μM peptide (SEQ ID NO: 2). In contrast, the normalized mean current at +40 mV doubled at 10 nM peptide (SEQ ID NO: 2), remained elevated up to about 10 μM peptide, and then fell dramatically at higher peptide concentrations (i.e. between about 10 μM and 50 μM peptide).

The specificity of the activation of cardiac RyR2 channels by the 20-mer peptide (SEQ ID NO: 2) was also tested. At 1 μM cis peptide NB (SEQ ID NO: 8) or 10 μM cis peptide NB, no modified channel activity was observed at +40 mV or −40 mV bilayer potential (n=3; FIG. 4). A scrambled sequence having the same isoelectric point as the test 20-mer peptide but a non-homologous sequence (i.e. Peptide A1S; SEQ ID NO: 9) reduced cardiac RyR2 channel activity when present at higher concentrations (i.e. 1 μM cis peptide or 10 μM cis peptide) in 2 out of 2 of the other bilayers (FIG. 4).

Data provide strong support that activation by the 20-mer peptide (SEQ ID NO: 2) reflects binding to the cardiac RyR2 channel.

Without limiting the present invention in any way, the ability of the 20-mer peptide (SEQ ID NO: 2) and peptide A1S at higher concentrations to inhibit cardiac RyR activity at +40 mV suggests, not unexpectedly, that the pore or its vestibule in both cardiac and skeletal RyRs contain a significant number of negative sites which can interact with positively charged residues in the peptides. The fact that inhibition persists with peptide A1S in the absence of activation provides further evidence that activation and inhibition depend on the 20-mer peptide (SEQ ID NO: 2) binding to two separate sites on the RyR2 channel.

The records show that the increase in single channel activity was not associated with any change in single channel conductance.

Channel activity also increased at 65 nM 20-mer peptide (SEQ ID NO: 2) when the bilayer potential was +40 mV (FIG. 3, panel B). However, the increase in channel activity was less than that observed for the same channel at −40 mV (FIG. 2, panel B).

Whilst channel activity at −40 mV bilayer potential increased at higher peptide concentration, channel opening at +40 mV began to decline at concentrations above about 100 nM peptide, and only a few brief channel openings were observed at 10 μM peptide, due to low conductance levels (FIG. 3, panel C). Without limiting the invention to any one theory or mode of action, the depression of activity at +40 mV, wherein channel openings are mostly due to sub maximal conductance, is thought to be attributable to binding of the peptide (SEQ ID NO: 2) to low affinity sites that are distinct from the sites at which the peptide binds during channel activation. These low affinity sites are probably within the channel pore.

Similar activation and inhibition of cardiac RyR2 channels in lipid bilayers was obtained in 8 out of 8 bilayer samples. Single channel analysis was not performed since most bilayers contained more than one channel. All samples had a higher frequency of opening at +40 mV, and at −40 mV, following peptide (SEQ ID NO: 2) addition to the cis chamber. Additionally, prolonged channel openings occurred at a potential of −40 mV in the presence of peptide, compared to that detected in the absence of peptide at 10⁻⁷M cis Ca³⁺.

The mean current (i.e. the average of all data points in two 30 s records, at each potential and each peptide concentration, divided by the number of channels seen in the recording) provided a measure of channel activity. Average normalized mean current is shown as a function of peptide concentration in FIG. 4. The integer I′p/I′c is the ratio of mean current in the presence of peptide to mean current before peptide addition to the chamber. A significant increase in mean current (˜2-fold) was detected in 10 nM peptide (SEQ ID NO: 2), at a bilayer potential of −40 mV or +40 mV. Mean current at −40 mV increased up to

### EXAMPLE 3

### Functional Analysis of DHPR 20-Mer Fragment Derivatives and Analogues

### Materials & Methods

Peptides

Four peptides were tested in this series of experiments, these being:
(i) the native DHPR 20-mer peptide (SEQ ID NO: 2);
(ii) the SEQ ID NO: 2 peptide in which Ser⁶⁸⁷ (residue 17) is replaced by an alanine residue to produce SEQ ID NO: 8;
(iii) the SEQ ID NO: 2 peptide in which Arg⁶⁸⁸ (residue 18) is replaced by an D isomer to produce SEQ ID NO: 9; and
(iv) the SEQ ID NO: 2 peptide in which Ser⁶⁸⁷ is mutated to alanine and Arg⁶⁸⁸ is replaced by a D isomer to produce SEQ ID NO: 10.
   Measurements of Ca²⁺ Release from Cardiac SR

Cardiac SR vesicles (50 μg of protein) were added to a cuvette, to a final volume of 2 ml of a solution containing (in mM): 100, KH₂PO₄ (pH=7); 4, MgCl₂; 1, Na₂ATP; 0.5, antipyrylazo III. Extravesicular [Ca²⁺] was monitored at 710 nm using either a Cary 50 or Cary 100 spectrophotometer. Identical experiments at 790 nm showed no changes in OD that were independent of changes in [Ca²⁺] which would alter the rate of Ca²⁺ release measured at 710 run. Vesicles were loaded with Ca²⁺ by 4 additions of 3 μl aliquots of 5 mM CaCl₂, to a final concentration of 7.5 μM Ca²⁺. Thapsigargin (200 nM) was then added to block the SR Ca²⁺ ATPase. Finally the peptides were added either alone, or with 20 μM Ca²⁺ or 2 mM caffeine. The rate of Ca²⁺ release in the presence of thapsigargin was measured just before addition of the activating agents and initial rates of Ca²⁺ release were measured immediately after addition of the activating agents. The specific blocker of RyR activity, 5 M ruthenium red, was then added to confirm that Ca²⁺ release was through RyR channels. The Ca⁺ ionophore A23187 (3 μg/ml) was added at the end of the experiment to determine the amount of Ca²⁺ remaining in the SR vesicles. When Ca²⁺ release was complete, the Ca²⁺ transient following addition of the ionophore, was indicative of the fraction of vesicles containing Ca²⁺ that could not be released through RyR channel (presumably vesicles from longitudinal SR that lack RyR channels). The results showed that only 10-20% of the cardiac SR preparation contained RyR regulated stores. Experiments were repeated on vesicles isolated from 3 different sheep heart preparations.

Single Channel Experiments.

The Ca²⁺ concentration in the cis solution was either buffered at 100 nM or was adjusted to 100 μM by adding an CaCl₂ to the cis solution in the absence of BAPTA.

### EXAMPLE 4

### Functional Analysis of DHPR 20-Mer Peptide Fragment Derivatives and Analogues

### Results

Summary

The example 4 experiments include the testing of all compounds on Ca²⁺ release from cardiac SR vesicles. In addition, single cardiac RyR channels were exposed to peptide SEQ ID NO: 9. All peptides significantly enhanced the rates of Ca⁺-activated Ca²⁺ release or caffeine-activated Ca²⁺ release. Peptides SEQ ID NOs: 9 and 10, at high concentrations of >30 μM marginally enhanced the rate of Ca²⁺ release in the absence of activating Ca²⁺ or caffeine, and also enhanced both Ca²⁺-activated Ca²⁺ release and caffeine-activated Ca²⁺ release. Peptide SEQ ID NO: 9 enhanced cardiac RyR activity in lipid bilayer experiments at low concentrations (1-10 nM), but blocked the channel pore in a voltage- and Ca²⁺-dependent manner at +40 mV at high concentrations. Accordingly, under appropriate physiological conditions, the dihydropyridine receptor fragment peptides are able to bind to, and activate, the cardiac Ca²⁺ release channel and Ca²⁺ release from cardiac SR.

Effects of the A Peptides on Ca²⁺ Release from Cardiac SR.

The ability of the peptides defined by SEQ ID NOs: 2, 8, 9 and 10 to release Ca²⁺ from cardiac SR, in the absence of additional activating factors, was determined. The peptides were added to the extravesicular solution and initial rates of Ca²⁺ release immediately following the addition of peptides were measured. Average data for the four peptides is shown in FIG. 5 (small symbols). A small increase in the rate of release was seen with all peptides at concentrations of 30 μM and 50 μM.

The effects of the peptides on Ca²⁺-activated Ca²⁺ release and caffeine-activated Ca²⁺ release were examined. Ca²⁺-activation is the principal in vivo mechanism of RyR activation during cardiac contraction. The Ca²⁺-activation mechanism is responsible for both Ca²⁺-activated and caffeine-activated Ca²⁺ release from SR vesicles, since the major effect of caffeine is to shift the Ca²⁺-activation curve to much lower Ca²⁺ concentrations. The resting Ca²⁺ concentration of ˜100 nM activates Ca⁺ release in the presence of caffeine. The initial rates of Ca²⁺ release induced by 20 μM Ca²⁺ and 2 mM caffeine were similar and were ˜10 μmoles of Ca²⁺ per mg of SR protein per min in these experiments. The effects of the peptides on Ca²⁺-induced Ca²⁺ release and caffeine-induced Ca²⁺ release were also similar and data obtained with the two methods of activation is grouped to ether in the average data shown in FIG. 5. Clearly, each of the four peptides was able to increase the Ca²⁺/caffeine-activated Ca²⁺ release from the cardiac SR vesicles when added to the extravesicular solution. In all experiments, Ca²⁺ release was terminated by addition of ruthenium red, indicating that release was through RyR channels. The maximum rate of Ca²⁺ release with each of the four peptides was 2 to 2.5-fold greater than control and was achieved at 20-30 μM peptide. There appeared to be a biphasic action with the four peptides in that the rates of Ca²⁺ release tended to fall at higher peptide concentrations. The peptides all showed similar potency in releasing Ca²⁺ from cardiac SR and in enhancing Ca²⁺/caffeine-activated Ca²⁺ release. It is possible that the release from non Ca²⁺/caffeine-activated channels was too small to pick up differences between peptides. Without limiting the invention in any way, this similarity between the effects of the peptides on Ca²⁺/caffeine-activated Ca²⁺ release may have reflected the fact that the RyR channels were close to maximally open with the activating agents alone and thus their capacity to be further activated by the peptides was limited.

Effects of Peptide SEQ ID NO: 9 on Single Cardiac RyR Channel Activity.

The action of SEQ ID NO: 9 on the activist single cardiac RyR channels in lipid bilayers was examined. The peptide was initially added to cytoplasmic (cis) side of cardiac RyR channels with 100 μM cis Ca²⁺. The peptide failed to activate channels (FIG. 6).

The reduction in the activity of cardiac RyR channels induced when peptide SEQ ID NO: 9 was added to the bilayer solution was greater at +40 mV than −40 mV and was thus similar to the voltage-dependent (i.e. current direction-dependent) block of skeletal RyR channels by peptide SEQ ID NO: 2. The reduction in activity is consistent with the positively charges SEQ ID NO: 9 peptide entering the ion channel and associating with negative charges within the pore. The block is enhanced when current flow is from cis to trans and carries the peptide into the pore, but is partially reversed when current flows from trans to cis and tends to carry the peptide out of the pore.

A cis Ca²⁺ concentration of 100 nM peptide, buffered with 2 mM BAPTA was used. Strong activation of the cardiac channels by peptide SEQ ID NO: 9 caused a 10-20 fold increase in relative mean current at both positive and negative potentials (FIG. 7). There was a significant increase in activity with only 10 nM peptide and maximum activation was observed with 100 nM peptide. A fall in the relative mean current at higher peptide concentrations (1 and 10 μM) at +40 mV was indicative of a residual blocking action of the peptide.

The effects of this peptide on average single channel parameters of cardiac RyRs was measured and average data is shown in FIG. 8. The peptide induced an ˜50-fold increase in the open probability −40 mV and an ˜10-fold increase at +40 m˜. This increase in activity was due to an 8-fold increase in the mean open time at both potentials and an 80- or 170-fold decrease in the mean closed times at +40 mV and −40 mV respectively, and a corresponding increase in the frequency of events. Thus the major effect on the peptide on channel gating is on the mean closed time.

Accordingly, the DHPR II-III loop SEQ ID NO: 2 peptide can activate cardiac RyR channels and Ca²⁺ release from cardiac SR vesicles. Not only does the native peptide release Ca²⁺, several other peptides with enhanced helical structure, and preferably containing the RKRRK sequence, exhibit the same action on Ca²⁺ release.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

### REFERENCES

Ahern, G. P., P. R. Junankar, and A. F. Dulhunty. 1994. FEBS Lett. 352:369-374.
Bhat, M. B., J. Zhao, H. Takeshima, and J. Ma. 1997. Biophys. J 73:1329-1336.
Chaudhari, N. 1992. J Biol Chem 1992 Dec. 25;267(36):25636-9 267:25636-25639.
Cheng, H., W. J. Lederer, and M. B. Cannell. 1993. Science 262:740-744.
Devereux, J., Haeberli, P. and Smithies, O. 1984. Nucl. Acids Res. 12: 387-395.
Drouet, B., L. Garcia, D. Simon-Chazottes, M. Mattei, J. Guenet, A. Schwartz, G. Varadi, and M. Pincon-Raymond. 1993. Mamm Genome 4:499-503.
Dulhunty, A. F., D. R. Layer, E. M. Gallant, M. G. Casarotto, S. M. Pace, and S. Curtis. 1999. Biophys. J. 77:189-203.
El-Hayek, R., B. Antoniu, J. Wang, S. L. Hamilton, and N. Ikemoto. 1995. J. Biol. Chem. 270:22116-22118.
Gomez, A., H. Valdivia, H. Cheng, M. Lederer, L. Santana, M. Cannell, S. McCune, R. Altschuld, and W. Lederer. 1997. Science 276:755-756.
Gurrola, G. B., C. Arevalo, R. Sreekumar, A. J. Lokuta, J. W. Walker, and H. H. Valdivia. 1999. J. Biol. Chem. 274:7879-7886.
Layer, D. R., L. D. Roden, G. P. Ahern, K. R. Eager, P. R. Junankar, and A. F. Dulhunty. 1995. J. Membr. Biol. 147:7-22.
Lu, X., L. Xu, and G. Meissner. 1994. J. Biol. Chem. 269:6511-6516.
McCall, E., L. V. Hryshko, V. M. Stiffel, D. M. Christensen, and D. M. Bers. 1996. J. Mol. Cell Cardiol. 28:79-93.
Needleman and Wunsch. 1970 J. Mol. Biol. 48: 443-453.
Niggli, E. 1999. Annu Rev Physiol 61:311-335.
O'Reilly, F. M. and M. Ronjat. 1999. Biophys. J. 76:A466-(abstract).
Osterland, J. C., S. M. Hayek, X. Xu, M. B. Bhat, and J. Ma. 1999. Biophys. J. 76:A467-(abstract).
Stem, M. D. and E. G. Lakatta. 1992. FASEB J. 6:3092-3100.
Tanabe, T., H. Takeshima, A. Mikami, V. Flockerzi, H. Takahashi, K. Kangawa, M. Kojima, H. Matsuo, T. Hirose, and S. Numa. 1987. Nature 328:313-318.
Tanabe, T., K. G. Beam, B. A. Adams, T. Niidome, and S. Numa. 1990. Nature 346:567-568.
Thompson, et al 1994. Nucl. Acids Res. 22: 4673-4680
Zaloga, G. P., P. R. Roberts, K. W. Black, M. Lin, G. Zapata-Sudo, R. T. Sudo, and T. E. Nelson. 1997. Am. J. Physiol. 272:H462-H468.
Zhou, J., L. Cribbs, J. Yi, R. Shirokov, E. Perez-Reyes, and E. Rios. 1998. J. Biol. Chem. 273:25503-25509.

1. A method for modulating the activity of a cardiac ryanodine receptor calcium channel comprising contacting a cardiac ryanodine channel with an amount of a fragment of a skeletal dihydropyridine receptor polypeptide or derivative, homologue or analogue sufficient to modulate the activity of said ryanodine channel.

2. The method according to claim 1 wherein said method comprises the additional step of determining the activity of said cardiac ryanodine calcium channel.

3. The method according to claim 1 or 2 wherein said modulation is upregulation.

4. The method according to claim 3 wherein said fragment is applied at a concentration in the range of about 1 nm to about 10 μM.

5. The method according to claim 1 or 2 wherein said modulation is down-regulation.

6. The method according to claim 5 wherein said fragment is applied at a concentration in excess of about 10 μM.

7. The method according to any one of claims 1-6 wherein said fragment comprises at least five contiguous amino acid residues of the peptide sequence:
Thr Ser Ala Gin Lys Xaa Xaa Xaa Xaa Glu Glu Xaa Xaa Arg Ser Lys
Xaa Xaa Xaa Xaa Xaa (SEQ ID NO:1) and the RKRRK motif.

8. The method according to claim 7 wherein said peptide sequence corresponds to any one of the following peptide sequences:

| | | |
|---|---|---|
| (i) | Thr Ser Ala Gln Lys Ala Lys Ala Glu Glu Arg Lys Arg Arg Lys Met Ser | (SEQ ID NO: 3) |
| | Lys Gly Leu | |
| (ii) | Thr Ser Ala Gln Lys Ala Lys Ala Glu Glu Arg Lys Arg Arg Lys Met Ser | (SEQ ID NO: 4) |
| | Arg Gly Leu | |
| (iii) | Thr Ser Ala Gln Lys Ala Lys Ala Glu Glu Arg Lys Arg Arg Lys Met Ser | (SEQ ID NO: 2) |
| | Arg Gly Leu | |
| (iv) | Thr Ser Ala Gln Lys Ala Lys Ala Glu Glu Arg Lys Arg Arg Lys Met Ala | (SEQ ID NO: 8) |
| | Arg Gly Leu | |
| (v) | Thr Ser Ala Gln Lys Ala Lys Ala Glu Glu Arg Lys Arg Arg Lys Met Ser | (SEQ ID NO: 9) |
| | Xaa Gly Leu | |
| (vi) | Thr Ser Ala Gln Lys Ala Lys Ala Glu Glu Arg Lys Arg Arg Lys Met Ala | (SEQ ID NO: 10) |
| | Xaa Gly Leu | |

or a functional derivative, homologue or amologue thereof.

9. The method according to claim 1-8, wherein said peptide sequence comprises at least 10 contiguous amino acid residues and more preferably at least 15-20 contiguous amino acid residues.

10. The method according to claim 9 wherein said fragment is a basic charged fragment.

11. A method for identifying a peptide or non-peptide modulator of a cardiac ryanodine calcium channel comprising:
(i) incubating an amount of a fragment of a skeletaldihydropyridine receptor polypeptide or a homologue, analogue or derivative thereof that modulates cardiac ryanodine channel activity in the presence of a functional cardiac ryanodine calcium channel under conditions appropriate for calcium channel activity to be modulated and determining the activity of the channel;
(ii) incubating a candidate peptide or non-peptide compound in the presence of said functional cardiac ryanodine calcium channel under conditions appropriate for calcium channel activity to be modulated by said dihydropyridine receptor polypeptide fragment or a homologue, analogue or derivative thereof and determining the activity of the channel; and
(iii) comparing the activity at (i) and (ii).

12. The method according to claim 11 wherein said modulation is up-regulation.

13. The method according to claim 12 wherein said fragment is applied at a concentration in the range of about 1 nm to about 10 μm.

14 The method according to claim 13, wherein said modulation is down-regulation.

15. The method according to claim 14, wherein said fragment is applied at a concentration in excess of about 10 μM.

16. The method according to any one of claims 11-15 wherein said fragment comprises the peptide sequence:

| | |
|---|---|
| Thr Ser Ala Gln Lys Xaa Xaa Xaa Xaa Glu Glu Xaa Xaa Arg Ser Lys | (SEQ ID NO: 1) |
| Xaa Xaa Xaa Xaa Xaa | |
| and | |
| the RKRRK motif. | |

17. The method according to any one of claims 11 to 16, wherein said peptide sequence corresponds to any one of the following peptide sequences:

| | | |
|---|---|---|
| (i) | Thr Ser Ala Gln Lys Ala Lys Ala Glu Glu Arg Lys Arg Arg Lys Met Ser | (SEQ ID NO: 3) |
| | Lys Gly Leu | |
| (ii) | Thr Ser Ala Gln Lys Ala Lys Ala Glu Glu Arg Lys Arg Arg Lys Met Ser | (SEQ ID NO: 4) |
| | Arg Gly Leu | |
| (iii) | Thr Ser Ala Gln Lys Ala Lys Ala Glu Glu Arg Lys Arg Arg Lys Met Ser | (SEQ ID NO: 2) |
| | Arg Gly Leu | |
| (iv) | Thr Ser Ala Gln Lys Ala Lys Ala Glu Glu Arg Lys Arg Arg Lys Met Ala | (SEQ ID NO: 8) |
| | Arg Gly Leu | |
| (v) | Thr Ser Ala Gln Lys Ala Lys Ala Glu Glu Arg Lys Arg Arg Lys Met Ser | (SEQ ID NO: 9) |
| | Xaa Gly Leu | |
| (vi) | Thr Ser Ala Gln Lys Ala Lys Ala Glu Glu Arg Lys Arg Arg Lys Met Ala | (SEQ ID NO: 10) |
| | Xaa Gly Leu | |

or a functional derivative, homologie or amalogue thereof.

18. The method according to any one of calims 11-17, wherein said peptide comprises at least 10 contiguous amino acid residues and more preferably at least 15-20 contiguous amino acid residues.

19. The method according to claim 18, wherein said fragment is a basic charged fragment.

20. A process comprising:
(i) identifying candidate agonists and antagonists of a cardiac ryanodine calcium channel;
(ii) determining those compounds at (i) that actually activate or inhibit the activity of a cardiac ryanodine channel;
(iii) determining which compounds at (ii) have higher binding affinities for said cardiac ryanodine calcium channel than any one of SEQ ID NOs: 1-10; and
(iv) optionally, determining the sites of interaction between those compounds at (iii) and said cardiac ryanodine calcium channel.

21. A method of determining whether a cardiac ryanodine channel is open or has a high channel open probability said method comprising contacting a cardiac ryanodine channel with an amount of a fragment of a skeletal dihydropyridine receptor polypeptide for a time and under conditions sufficient for binding to ryanodine to occur and determining the binding of said peptide to ryanodine, wherein binding of said peptide to ryanodine indicates a high channel open probability and wherein non-specific peptide binding of peptide to the channel pore indicates a low channel open probability.

22. The method according to claim 25 wherein said fragment comprises at least 5 contiguous amino acid residues of the peptide sequence:
Thr Ser Ala Gin Lys Xaa Xaa Xaa Xaa Glu Glu Xaa Xaa Arg Ser Lys
Xaa Xaa Xaa Xaa Xaa (SEQ ID NO:1) and the RKRRK motif.

23. The method according to claim 22, wherein said peptide sequence corresponds to any one of the following peptide sequence:

| | | |
|---|---|---|
| (i) | Thr Ser Ala Gln Lys Ala Lys Ala Glu Glu Arg Lys Arg Arg Lys Met Ser | (SEQ ID NO: 3) |
| | Lys Gly Leu | |
| (ii) | Thr Ser Ala Gln Lys Ala Lys Ala Glu Glu Arg Lys Arg Arg Lys Met Ser | (SEQ ID NO: 4) |
| | Arg Gly Leu | |
| (iii) | Thr Ser Ala Gln Lys Ala Lys Ala Glu Glu Arg Lys Arg Arg Lys Met Ser | (SEQ ID NO: 2) |
| | Arg Gly Leu | |
| (iv) | Thr Ser Ala Gln Lys Ala Lys Ala Glu Glu Arg Lys Arg Arg Lys Met Ala | (SEQ ID NO: 8) |
| | Arg Gly Leu | |
| (v) | Thr Ser Ala Gln Lys Ala Lys Ala Glu Glu Arg Lys Arg Arg Lys Met Ser | (SEQ ID NO: 9) |
| | Xaa Gly Leu | |
| (vi) | Thr Ser Ala Gln Lys Ala Lys Ala Glu Glu Arg Lys Arg Arg Lys Met Ala | (SEQ ID NO: 10) |
| | Xaa Gly Leu | |

or a functional derivative, homologue or amalogue thereof.

24. The method according to any one of claims 21-23 wherein said peptide comprises at least 10 contiguous amino acid residues and more preferably at least 15-20 contiguous amino acid residues.

25. The method according to claim 24 wherein said fragment is a basic charged fragment.

26. A method of treatment of cardiac dysfunction in a human or animal subject comprising administering an effective amount of a fragment of a skeletal dihydropyridine receptor polypeptide comprising a RKRRK motif for a time and under conditions sufficient for enhanced cardiac contraction to occur thereby rectifying said cardiac dysfunction.

27. The method according to claim 30 wherein said cardiac dysfunction is myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug (e.g. cocaine)-induced cardiomyopathy, infarction, dysrhythmia, congestive heart failure, or heart attack.

28. The method according to claim 32 or 33 wherein said fragment comprises at least 5 contiguous amino acid residues of the peptide sequence:

| | |
|---|---|
| Thr Ser Ala Gln Lys Xaa Xaa Xaa Xaa Glu Glu Xaa Xaa Arg Ser Lys | (SEQ ID NO: 1) |
| Xaa Xaa Xaa Xaa Xaa | |
| and | |
| the RKRRK motif. | |

29. The method according to claim 34 wherein said peptide sequence corresponds to any one of the following peptide sequences:

| | | |
|---|---|---|
| (i) | Thr Ser Ala Gln Lys Ala Lys Ala Glu Glu Arg Lys Arg Arg Lys Met Ser | (SEQ ID NO: 3) |
| | Lys Gly Leu | |
| (ii) | Thr Ser Ala Gln Lys Ala Lys Ala Glu Glu Arg Lys Arg Arg Lys Met Ser | (SEQ ID NO: 4) |
| | Arg Gly Leu | |
| (iii) | Thr Ser Ala Gln Lys Ala Lys Ala Glu Glu Arg Lys Arg Arg Lys Met Ser | (SEQ ID NO: 2) |
| | Arg Gly Leu | |
| (iv) | Thr Ser Ala Gln Lys Ala Lys Ala Glu Glu Arg Lys Arg Arg Lys Met Ala | (SEQ ID NO: 8) |
| | Arg Gly Leu | |
| (v) | Thr Ser Ala Gln Lys Ala Lys Ala Glu Glu Arg Lys Arg Arg Lys Met Ser | (SEQ ID NO: 9) |
| | Xaa Gly Leu | |
| (vi) | Thr Ser Ala Gln Lys Ala Lys Ala Glu Glu Arg Lys Arg Arg Lys Met Ala | (SEQ ID NO: 10) |
| | Xaa Gly Leu | |

or a functional derivative, homologue or amalogue thereof.

30. The method according to any one of claims 26-29 wherein said peptide comprises at least 10 contiguous amino acid residues and more preferably at least 15-20 contiguous amino acid residues.

31. The method according to claim 38 wherein said fragment is a basic charged fragment.

32. The use of a fragment of a skeletal dihydropyridine receptor peptide comprising at least 5 contiguous amino acid residues of the peptide set forth in any one of SEQ ID NOs: 1-10 and the RKRRK motif or a functional homologue, analogue, or derivative thereof, to modify the activity of cardiac ryanodine calcium channel, thereby modifying defective calcium signaling.

33. Use according to claim 32 wherein said peptide comprises at least 10 contiguous amino acid residues and more preferably at least 15-20 contiguous amino acid residues.

34. Use according to claim 32 or 33 wherein said defective calcium signaling induces chronic hypertrophy, dilated cardiac myopathy or heart failure.

35. Use according to claim 32 or 33 wherein said peptide or homologue, analogue or derivative thereof is administered as a dosage that can enhance contractile force, and further increase intracellular calcium concentration (i.e. [Ca²⁺]ᵢ) during systole, and further decrease [Ca²⁺]ᵢ during diastole.

36. Use according to claim 35 wherein said peptide or a homologue, analogue or derivative thereof induces at least about a 3% or 5% increase in systolic [Ca²⁺]ᵢ relative to the systolic [Ca²⁺]ᵢ.

37. Use according to claim 35 wherein said peptide or a homologue, analogue or derivative thereof induces at least about a 3% or 5% decrease in diastolic [Ca²⁺]ᵢ relative to the diastolic [Ca²⁺]ᵢ.

38. Use according to either claim 36 or 37 wherein said systolic [Ca²⁺]ᵢ is increased, or diastolic [Ca²⁺]ᵢ is decreased, by at least about 10% or 15%, and still more preferably by at least about 20%, 25%, 30%, 40% or 50%, relative to the systolic [Ca²⁺]ᵢ or diastolic [Ca²⁺]ᵢ respectively.

39. Use according to any one of claims 32-38 wherein said administered peptide induces an improvement in the efficiency of cardiac contraction.

40. Use according to claim 39 wherein said cardiac contraction is enhanced by inducing at least about a 5% or 10% increase in preload-recruitable stroke work at within 0.5-1.0 hr following administration.

41. Use according to claim 40 wherein said cardiac contraction is enhanced by about 15%, 20%, 30%, 40%, 50%, 55%, 60% or 70%.

42. The use of a fragment of a skeletal dihydropyridine receptor polypeptide comprising at least 5 contiguous amino acid residues of the peptide set forth in any one of SEQ ID NOs: 1-10 and a RKRRK motif or a homologue, analogue or derivative thereof in the manufacture of a medicament for the treatment of cardiac dysfunction in a human or animal subject.

43. Use according to claim 41 wherein said cardiac dysfunction is myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug (e.g. cocaine)-induced cardiomyopathy, infarction, dysrhythmia, congestive heart failure, or heart attack.

44. Use according to claim 50 or 51 wherein said peptide comprises at least 10 contiguous amino acid residues and more preferably at least 15-20 contiguous amino acid residues.

45. A pharmaceutical composition comprising a fragment of a skeletal dihydropyridine receptor polypeptide, which peptide comprises at least about 5 contiguous amino acid residues of the peptide set forth in any one of SEQ ID NOs: 1-10 amd a RKRRK motif or a functional homologue, analogue or derivative thereof together with one or more pharmaceutically acceptable carriers and/or diluents.

46. The pharmaceutical composition according to claim 45 wherein said peptide comprises at least 10 contiguous amino acid residues and more preferably at least 15-20 contiguous amino acid residues.

47. The pharmaceutical composition of claim 45 or 46 when used according to the method of any one of claims 25-30.


SEQUENCE LISTING (13 sequences)

SEQ ID NO: 1 - protein, 20 aa, Artificial sequence
  Description of artificial sequence  synthetic consensus DHPR 20-mer peptide
TSAQKXXXEEXXRXKXXXXX

SEQ ID NO: 2 - protein, 20 aa, Artificial sequence
  Description of artificial sequence  synthetic human skeletal DHPR 20-mer peptide
TSAQKAKAEERKRRKMSRGL

SEQ ID NO: 3 - protein, 20 aa, Artificial sequence
  Description of artificial sequence  synthetic murine skeletal DHPR 20-mer peptide
TSAQKAKAEERKRRKMSKGL

SEQ ID NO: 4 - protein, 20 aa, Artificial sequence
  Description of artificial sequence  synthetic rabbit skeletal DHPR 20-mer peptide
TSAQKAKAEERKRRKMSRGL

SEQ ID NO: 5 - protein, 20 aa, Artificial sequence
  Description of artificial sequence  synthetic rabbit cardiac DHPR 20-mer peptide
TSAQKEEEEEKERKKLARTA

SEQ ID NO: 6 - protein, 20 aa, Artificial sequence
  Description of artificial sequence  synthetic rat cardiac DHPR 20-mer peptide
TSAQKEEEEEKERKKLARTA

SEQ ID NO: 7 - protein, 20 aa, Artificial sequence
  Description of artificial sequence  synthetic bullfrog skeletal DHPR 20-mer peptide
TSAQKAKAEERKRKKLARAN

SEQ ID NO: 8 - protein, 20 aa, Artificial sequence
  Description of artificial sequence  synthetic derivative of human skeletal DHPR 20-mer peptide
TSAQKAKAEERKRRKMARGL

SEQ ID NO: 9 - protein, 20 aa, Artificial sequence
  Description of artificial sequence  analogue of human skeletal SHPR 20-mer peptide
TSAQKAKAEERKRRKMSXGL

SEQ ID NO: 10 - protein, 20 aa, Artificial sequence
  Description of artificial sequence  analogue of human skeletal DHPR 20-mer peptide
TSAQKAKAEERKRRKMAXGL

SEQ ID NO: 11 - protein, 20 aa, Artificial sequence
  Description of artificial sequence  synthetic peptide NB
GLPDKTEEEKSVMAKKLEQK

SEQ ID NO: 12 - protein, 20 aa, Artificial sequence
  Description of artificial sequence synthetic peptide A1S
TRKSRLARGQKAKAKSEMRE

SEQ ID NO: 13 - protein, 37 aa, Artificial sequence
  Description of artificial sequence  synthetic RyR2 peptide
FRAEKTYAVKAGRWYFEFEAVTSGDMRVGWSRPGCQP